(19) United States
(12) Patent Application Publication
(10) Pub. No.: US 2011/0144321 A1
(43) Pub. Date: Jun. 16, 2011

(54) SYNTHETIC INTERMEDIATES AND PROCESSES

(75) Inventors: Pooran Chand, Birmingham, AL (US); Minwan Wu, Birmingham, AL (US); Pravin L. Kotian, Birmingham, AL (US); V. Satish Kumar, Birmingham, AL (US); Tsu-Hsing Lin, Birmingham, AL (US)

(73) Assignee: BIOCRYST PHARMACEUTICALS, INC., Birmingham, AL (US)

(21) Appl. No.: 13/055,359

(22) PCT Filed: Jul. 22, 2009
(86) PCT No.: PCT/US2009/051402

§ 371 Date: Jan. 21, 2011

(51) Int. Cl. C07H 7/06; C07D 491/048; C07F 7/10; C07F 3/06; C07F 3/08; C07F 1/08; C07D 307/68; C07D 239/36; C07H 1/00

(52) U.S. Cl. 536/55; 544/278; 544/229; 544/226; 544/225; 549/480; 544/298; 536/124

(57) ABSTRACT

The invention provides novel synthetic intermediates and processes that can be used to prepare compounds of formula (I), wherein R¹ has any of the values described herein and R² is a nucleoside sugar group.

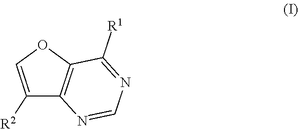

### CROSS-REFERENCE TO RELATED APPLICATION(S)

This patent application claims the benefit of priority of U.S. application Ser. No. 61/082,694, filed Jul. 22, 2008, which application is herein incorporated by reference.

### BACKGROUND OF THE INVENTION

International patent application publication number WO 2006/050161 describes compounds that are reported to be inhibitors of viral RNA and DNA polymerases. The compounds include compounds of formula (I):

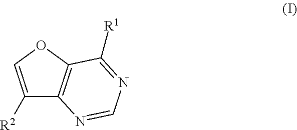

wherein R¹ is selected from a group of substituents, and R² is a nucleoside sugar group.

### SUMMARY OF CERTAIN EMBODIMENTS OF THE INVENTION

The present invention provides novel synthetic intermediates and processes that can be used to prepare compounds of formula (I). Accordingly, in one embodiment, the invention provides a compound of the invention, which is a compound of formula II:

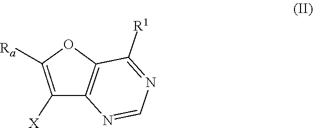

wherein:

R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;

n is 0-5;

R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;

Rₐ is H or Si(CH₃)₃; and

X is Br or I; or a protected analog thereof.

In another embodiment the invention provides a compound of formula III:

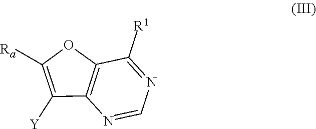

wherein:

R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;

n is 0-5;

R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;

Rₐ is H or Si(CH₃)₃; and

Y is a metallic group.

The invention also provides novel compounds of formulae (a)-(bn) described herein as well as processes for preparing compounds of formulae (a)-(bn) described herein.

The invention also provides novel compounds of formulae (1-1 to 14-2) described herein as well as processes for preparing compounds of formulae (1-1 to 14-2) described herein.

### DETAILED DESCRIPTION

The term “alkyl” as used herein refers to alkyl groups having from 1 to 6 carbon atoms. This term is exemplified by groups such as methyl, ethyl, n-propyl, iso-propyl, n-butyl, t-butyl, n-pentyl and the like. In a specific embodiment, the alkyl groups have from 1-4 carbon atoms and are referred to as lower alkyl.

The term “substituted alkyl” as used herein refers to an alkyl group having from 1 to 3 substituents, said substituents being selected from the group consisting of alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic.

The terms “alkenyl” or “alkene” as used herein refers to an alkenyl group having from 2 to 10 carbon atoms and having at least 1 site of alkenyl unsaturation. Such groups are exemplified by vinyl(ethen-1-yl), allyl, but-3-en-1-yl, and the like.

The term “substituted alkenyl” as used herein refers to alkenyl groups having from 1 to 3 substituents, said substituents being selected from those describe above for a substituted alkyl.

The term “alkynyl” or “alkyne” as used herein refers to an alkynyl group having from 2-10 carbon atoms and having at least 1 site of alkynyl unsaturation. Such groups are exemplified by, but not limited to, ethyn-1-yl, propyn-1-yl, propyn-2-yl, 1-methylprop-2-yn-1-yl, butyn-1-yl, butyn-2-yl, butyn-3-yl, and the like.

The term “substituted alkynyl” as used herein refers to alkynyl groups having from 1 to 3 substituents, said substituents being selected those describe above for a substituted alkyl.

The term “alkoxy” refers to the group alkyl-O—.

The term “substituted alkoxy” as used herein refers to the group substituted alkyl-O—.

The term “acyl” as used herein refers to the groups alkyl-C(O)—, alkenyl-C(O)—, alkynyl-C(O) cycloalkyl-C(O)—, aryl-C(O)—, heteroaryl-C(O)—, and heterocyclic-C(O).

The term “substituted acyl” as used herein refers to the groups substituted alkyl-C(O)—, substituted alkenyl-C(O)—, substituted alkynyl-C(O)—, substituted cycloalkyl-C(O)—, substituted aryl-C(O)—, substituted heteroaryl-C(O), and substituted heterocyclic-C(O)—.

The term “acylamino” as used herein refers to the group —C(O)NZ₁Z₂ where each Z₁ and Z₂ are independently selected from the group consisting of hydrogen, alkyl, substituted alkyl, alkenyl, substituted alkenyl, alkynyl and substituted alkynyl, and the substituents described above in the definition of substituted alkyl.

The term “acyloxy” as used herein refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—.

The term “oxyacyl” as used herein refers to the groups alkyl-OC(O) substituted alkyl-OC(O)—, alkenyl-OC(O)—, substituted alkenyl-OC(O) alkynyl-OC(O)—, substituted alkynyl-OC(O)—, aryl-OC(O)—, substituted aryl-OC(O)—, cycloalkyl-OC(O)—, substituted cycloalkyl-OC(O)—, heteroaryl-OC(O) substituted heteroaryl-OC(O)—, heterocyclic-OC(O)—, and substituted heterocyclic-OC(O)—.

The term “amino” as used herein refers to the group —NH₂. The term “substituted amino” as used herein refers to the group —NZ₁Z₂ where Z₁ and Z₂ are as described above in the definition of acylamino, provided that Zᵢ and Z₂ are both not hydrogen.

The term “aminoacyl” as used herein refers to the groups —NZ₃C(O)alkyl, —NZ₃C(O)substituted alkyl, —NZ₃C(O)cycloalkyl, —NZ₃C(O)substituted cycloalkyl, —NZ₃C(O)alkenyl, —NZ₃C(O)substituted alkenyl, —NZ₃C(O)alkynyl, —NZ₃C(O)substituted alkynyl, —NZ₃C(O)aryl, —NZ₃C(O)substituted aryl, —NZ₃C(O)heteroaryl, —NZ₃C(O)substituted heteroaryl, —NZ₃C(O)heterocyclic, and —NZ₃C(O)substituted heterocyclic, where each Z₃ is hydrogen or alkyl.

The term “aryl” as used herein refers to a monovalent aromatic cyclic group of from 6 to 14 carbon atoms having a single ring (e.g., phenyl) or multiple condensed rings (e.g., naphthyl or anthryl) which condensed rings may or may not be aromatic. Exemplary aryls include, but are not limited to, phenyl and naphthyl.

The term “substituted aryl” as used herein refers to aryl groups which are substituted with from 1 to 3 substituents selected from alkyl, substituted alkyl, alkenyl, substituted alkenyl, alkynyl and substituted alkynyl, and those substituents described above in the definition of substituted alkyl.

The term “aryloxy” as used herein refers to the group aryl-O— that includes, by way of example but not limitation, phenoxy, naphthoxy, and the like.

The term “substituted aryloxy” as used herein refers to substituted aryl-O-groups.

The term “carboxyl” as used herein refers to —COOH or salts thereof.

The term “carboxyl esters” as used herein refers to the groups-C(O)O-alkyl, —C(O)O-substituted alkyl, —C(O)O-aryl, and —C(O)O-substituted aryl.

The term “cycloalkyl” as used herein refers to a saturated or unsaturated cyclic hydrocarbon ring systems, such as those containing 1 to 3 rings and 3 to 7 carbons per ring. Exemplary groups include but are not limited to cyclopropyl, cyclobutyl, cyclopentyl, cyclohexyl, cycloheptyl, and adamantyl.

The term “substituted cycloalkyl” as used herein refers to a cycloalkyl having from 1 to 5 substituents selected from the group consisting of oxo (═O), thioxo (═S), alkyl, substituted alkyl, and those substituents described in the definition of substituted alkyl.

The term “cycloalkoxy” as used herein refers to —O-cycloalkyl groups.

The term “substituted cycloalkoxy” as used herein refers to —O-substituted cycloalkyl groups.

The term “formyl” as used herein refers to HC(O)—.

The term “halogen” as used herein refers to fluoro, chloro, bromo and iodo.

The term “heteroaryl” as used herein refers to an aromatic group of from 1 to 10 carbon atoms and 1 to 4 heteroatoms selected from the group consisting of oxygen, nitrogen, sulfur in the ring. The sulfur and nitrogen heteroatoms atoms may also be present in their oxidized forms. Such heteroaryl groups can have a single ring (e.g., pyridyl or furyl) or multiple condensed rings (e.g., indolizinyl or benzothienyl) wherein the condensed rings may or may not be aromatic and/or contain a heteroatom. Exemplary heteroaryl groups include, but are not limited to, heteroaryls include pyridyl, pyrrolyl, thienyl, indolyl, thiophenyl, and furyl.

The term “substituted heteroaryl” as used herein refers to heteroaryl groups that are substituted with from 1 to 3 substituents selected from the same group of substituents defined for substituted aryl.

The term “heteroaryloxy” as used herein refers to the group —O-heteroaryl.

The term “substituted heteroaryloxy” as used herein refers to the group —O-substituted heteroaryl.

The term “heterocycle” or “heterocyclic” or “heterocycloalkyl” refers to a saturated or unsaturated group (but not heteroaryl) having a single ring or multiple condensed rings, from 1 to 10 carbon atoms and from 1 to 4 hetero atoms selected from the group consisting of nitrogen, oxygen, sulfur, within the ring wherein, in fused ring systems, one or more the rings can be cycloalkyl, aryl or heteroaryl provided that the point of attachment is through the heterocyclic ring. The sulfur and nitrogen heteroatoms atoms may also be present in their oxidized forms.

The term “substituted heterocycle” or “substituted heterocyclic” or “substituted heterocycloalkyl” refers to heterocycle groups that are substituted with from 1 to 3 of the same substituents as defined for substituted aryl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

The term “heterocyclyloxy” as used herein refers to the group —O-heterocyclic.

The term “substituted heterocyclyloxy” as used herein refers to the group —O-substituted heterocyclic.

The term “phosphate” as used herein refers to the groups —OP(O)(OH)₂ (monophosphate or phospho), —OP(O)(OH)OP(O)(OH)₂ (diphosphate or diphospho) and —OP(O)(OH)OP(O)(OH)OP(O)(OH)₂ (triphosphate or triphospho) or salts thereof including partial salts thereof. It is understood that the initial oxygen of the mono-, di-, and triphosphate may include the oxygen atom of a sugar.

The term “phosphate esters” as used herein refers to the mono-, di- and tri-phosphate groups described above wherein one or more of the hydroxyl groups is replaced by an alkoxy group.

The term “phosphonate” refers to the groups —OP(O)(Z₄)(OH) or —OP(O) (Z₄)(OZ₄) or salts thereof including partial salts thereof, wherein each Z₄ is independently selected from hydrogen, alkyl, substituted alkyl, carboxylic acid, and carboxyl ester. It is understood that the initial oxygen of the phosphonate may include the oxygen of a sugar.

The term “thiol” as used herein refers to the group —SH.

The term “thioalkyl” or “alkylthioether” or “thioalkoxy” refers to the group-S-alkyl.

The term “substituted thioalkyl” or “substituted alkylthioether” or “substituted thioalkoxy” refers to the group —S-substituted alkyl.

The term “thiocycloalkyl” as used herein refers to the group —S-cycloalkyl.

The term “substituted thiocycloalkyl” as used herein refers to the group —S-substituted cycloalkyl.

The term “thioaryl” as used herein refers to the group —S-aryl.

The term “substituted thioaryl” as used herein refers to the group —S-substituted aryl.

The term “thioheteroaryl” as used herein refers to the group —S-heteroaryl.

The term “substituted thioheteroaryl” as used herein refers to the group —S-substituted heteroaryl.

The term “thioheterocyclic” as used herein refers to the group —S-heterocyclic.

The term “substituted thioheterocyclic as used herein refers to the group —S-substituted heterocyclic.

The term “amino acid sidechain” refers to the Z₇ substituent of α-amino acids of the formula Z₆NHCH(Z₇)COOH where Z₇ is selected from the group consisting of hydrogen, alkyl, substituted alkyl and aryl and Z₆ is hydrogen or together with Z₇ and the nitrogen and carbon atoms bound thereto respectively form a heterocyclic ring. In one embodiment, the α-amino acid sidechain is the sidechain one of the twenty naturally occurring L amino acids.

Sugars described herein may either be in D or L configuration.

The following schemes illustrate synthetic schemes and intermediates that are useful for preparing compounds of formula (I). In the following schemes, the designation (P) is used to signify one or more protecting groups on a synthetic intermediate.

The term “hydroxy protecting group” includes any group that can suitably protect a hydroxy group during a given reaction sequence. A variety of such groups are known (see for example Grene. T. E., Wuts, P. G., “Protective Groups in Organic Synthesis,” John Wiley & Sons, Inc., 3^{rd} Edition, 1999). A particular group of hydroxy protecting groups includes the following.
1. Ethers
   substituted methyl, such as methoxymethyl, benzyloxymethyl, p-methoxybenzyloxymethyl, t-butoxymethyl, 2,2,2-trichloroethoxymethyl, tetrahydropyranyl, 1,4-dioxan-2-yl, tetrahydrofuranyl, etc.
   substituted ethyl, such as 1-ethoxyethyl, 1-methyl-1-methoxymethyl, 2,2,2-trichloroethyl, t-butyl, allyl, p-methoxyphenyl, benzyl, etc.
   substituted benzyl, such as p-methoxybenzyl, p-halobenzyl, 2,6-dichlorobenzyl, 2,4-dichlorobenzyl, triphenylmethyl, diphenylmethyl, 9-anthryl, etc.
   silyl, such as trimethylsilyl, triethylsilyl, t-butyldimethylsilyl, t-butyldiphenylsilyl, diphenylmethylsilyl, etc.
2. Esters, such as formate, acetate, chloroacetate, trichloroacetate, trifluoroacetate, pivaloate, benzoate, etc.
3. Carbonates, such as methyl, ethyl, 2,2,2-trichloroethyl, isobutyl, allyl, vinyl, benzyl, etc.

The term “amino protecting group” includes any group that can suitably protect an amine group during a given reaction sequence. A variety of such groups are known (see for example Grene. T. E., Wuts, P. G., “Protective Groups in Organic Synthesis,” John Wiley & Sons, Inc., 3^{rd} Edition, 1999). A particular group of amino protecting groups includes the following.
1. Carbamates, such as methyl, ethyl, 9-fluorenylmethyl, 2,2,2-trichloroethyl, t-butyl, vinyl, allyl, benzyl, p-methoxybenzyl, 2,4-dichlorobenzyl, etc.
2. Amides, such as formyl, acetyl, piconyl, benzoyl, trichloroacetyl, trifluoroacetyl, etc.

The term “metallic group” includes any metal atom or metal complex that can be used to prepare a reactive species. For example, the term metallic group includes Li, and metal complexes that comprise Cu, Zn, Cd, and Mg.

The term “electrophilic nucleoside sugar group precursor” is a compound that can react with a nucleophile to provide compound that comprises a nucleoside sugar group or to provide a compound that can be further elaborated (e.g. deoxygenated) to provide a compound that comprises a nucleoside sugar group. For example, the term includes a compound of formula R²—Z; wherein R² is a nucleoside sugar group, and Z is a suitable leaving group. The term also includes a compound of formula R²(═O), wherein R² is a nucleoside sugar group. Such a compound can react with a nucleophile to provide an intermediate alcohol that can be deoxygenated to provide a compound that comprises a nucleoside sugar group. The term also includes an epoxide of formula R²(>O), wherein R² is a nucleoside sugar group. Such a compound can react with a nucleophile to directly provide a compound that comprises a nucleoside sugar group, or such a compound can react with a nucleophile to provide an intermediate alcohol that can be deoxygenated to provide a compound that comprises a nucleoside sugar group.

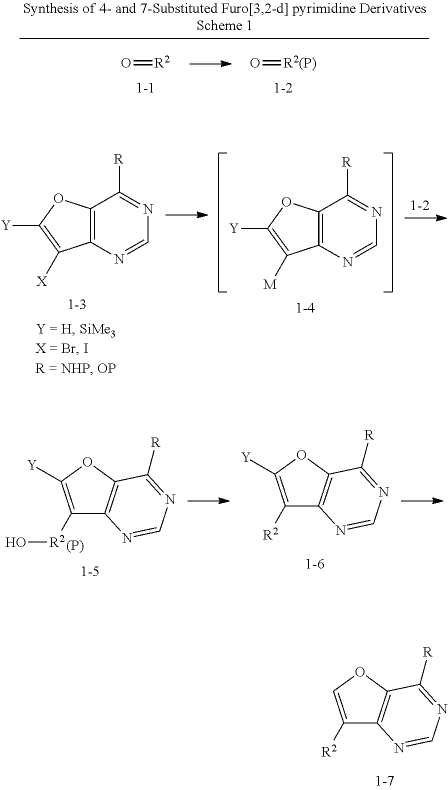

The synthesis of compounds represented by compound 1-7 is described in the above scheme. Compound 1-1, prepared by the literature procedures, is suitably protected by the methods given in Grene. T. E., Wuts, P. G., “Protective Groups in Organic Synthesis,” John Wiley & Sons, Inc., 3^{rd} Edition, 1999. The resultant compound 1-2 is one of the starting materials for the preparation of compound 1-7. The synthesis of other starting materials, compound 1-3 and intermediates, is described in the following schemes.

Compound 1-3 is treated with organometallic reagents like n-Buli, sec-Buli, tert-Buli, dialkylcuprate, dialkylzinc, dialkylcadmium, any alkylgrignard reagent, Mg, or any transition metal reagent to generate the reactive species 1-4, which upon treatment with compound 1-2 at a suitable temperature and in a suitable solvent yields the intermediate 1-5. The hydroxyl group in compound 1-5 is deoxygenated by reacting with Lewis acids like BF₃-Et₂O, etc., and reducing reagents such as triethylsilylhydride, boron hydrides such as sodium borohydride, lithium borohydride, sodium cyanoborohydride, etc., to give compound 1-6. If the isomers are obtained, the desired isomer is separated by suitable chromatographic procedures. The deprotection of protecting groups in compound 1-6 is accomplished by a number of methods found in Grene. T. E., Wuts, P. G., “Protective groups in Organic Synthesis,” John Wiley & Sons, Inc., 3^{rd} Edition, 1999 and compound 1-7 is obtained.

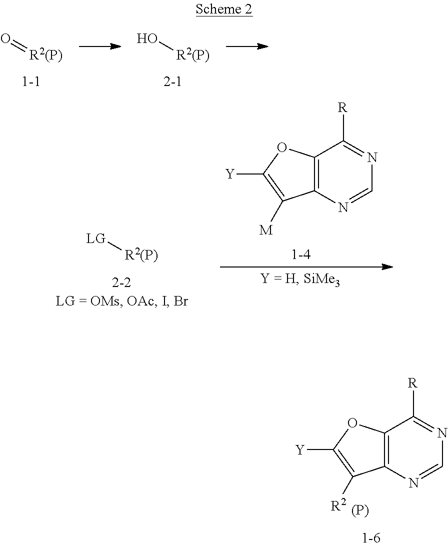

This scheme is particularly useful when 1-1 is a suitable lactone. Lactone 1-1 is partially reduced using reducing agents like diisobutyl aluminum hydride, vitride or like reagents to give compound 2-1. The hydroxyl group in compound 2-1 is converted to a suitable leaving group, such as acetate, which is obtained by the reaction of acetic anhydride in pyridine. The resultant compound 2-2 is treated with Lewis acid, like stanic chloride, BF₃-Et₂O, etc., and is reacted with compound 1-4 to give compound 1-6. The methods are reported in J. Med. Chem. 33, 2750-2755 (1990).

A representative lactone of formula 1-1 is a compound of the following formula:

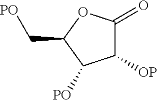

wherein each P independently a protecting group.

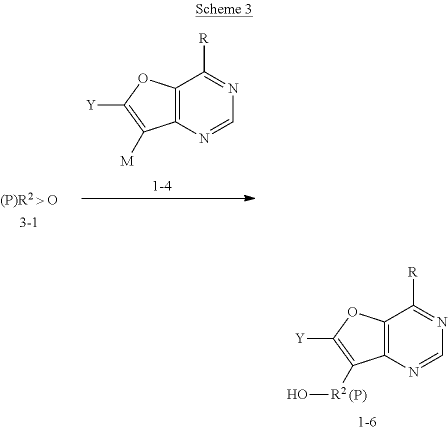

Yet another alternative method of preparation of compound 1-6 is described in Scheme 3. This method is particularly useful when there is one hydroxyl group present on the carbon to be attached to furopyrimidine and another hydroxyl on the adjacent carbon atom. These two hydroxyl groups are converted to an epoxide as shown in example 3-1, through known literature methods. The resultant epoxide 3-1 on reaction with compound 1-4 under suitable conditions produces the desired compound 1-6.

Representative epoxides of formula 3-1 include compounds of the following formulae:

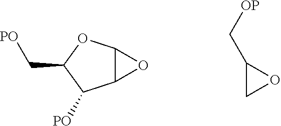

wherein each P independently a protecting group.

### Synthesis of Compound 1-3:

All of the three schemes above have used compound 1-3 or compound 1-4 as one of the starting materials. Compound 1-4 can be generated from compound 1-3 and the synthesis of compound 1-3 and intermediates is described in the following schemes.

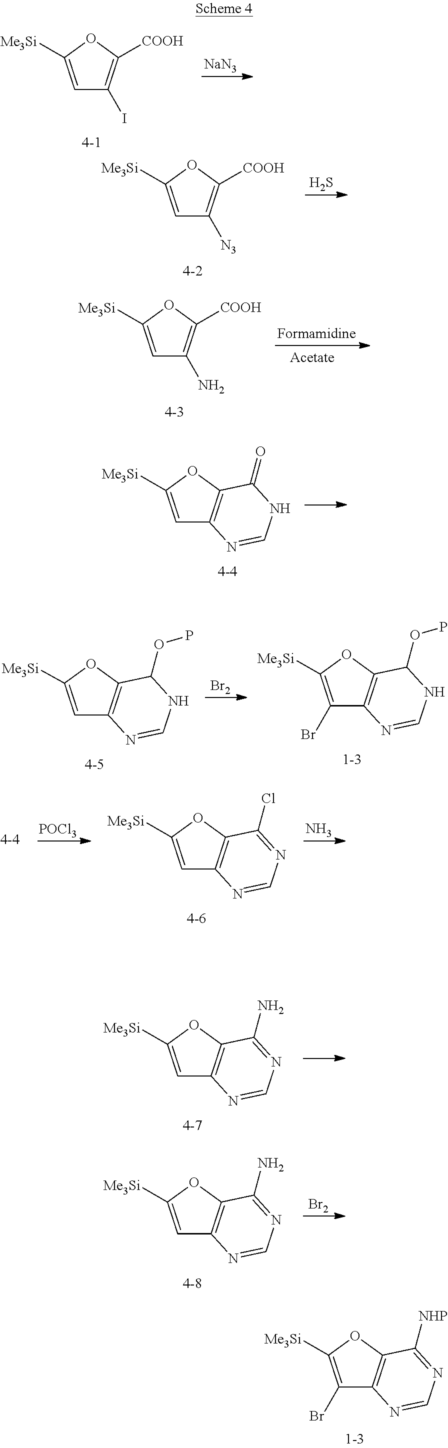

Preparation of Compound 1-3, when Y═SiMe₃, X═Br, R═NHP or OP:

The starting material, 3-iodo-5-trimethylsilyl-furan-2-carboxylic acid 4-1 can be prepared from the literature procedures (M. Takahashi et al., Heterocycles, 1867-1882 (1993)). Compound 4-1 is reacted with sodium azide to give compound 4-2, which upon reduction generates compound 4-3. Further cyclization of compound 4-3 to yield compound 4-4 is achieved through formamidine salt. The hydroxyl of compound 4-4 is protected to give compound 4-5 and bromination results in the required compound 1-3.

Compound 4-4 is converted to protected amino derivative also where R is NHR in compound 1-3. This is achieved by reacting compound 4-4 with POCl₃ to generate chloro compound 4-6. This resultant chloro compound is converted to compound 4-7 upon ammonia reaction. Further protection of amino group and bromination is obtained at the required place with suitable reagents to generate compound 1-3.

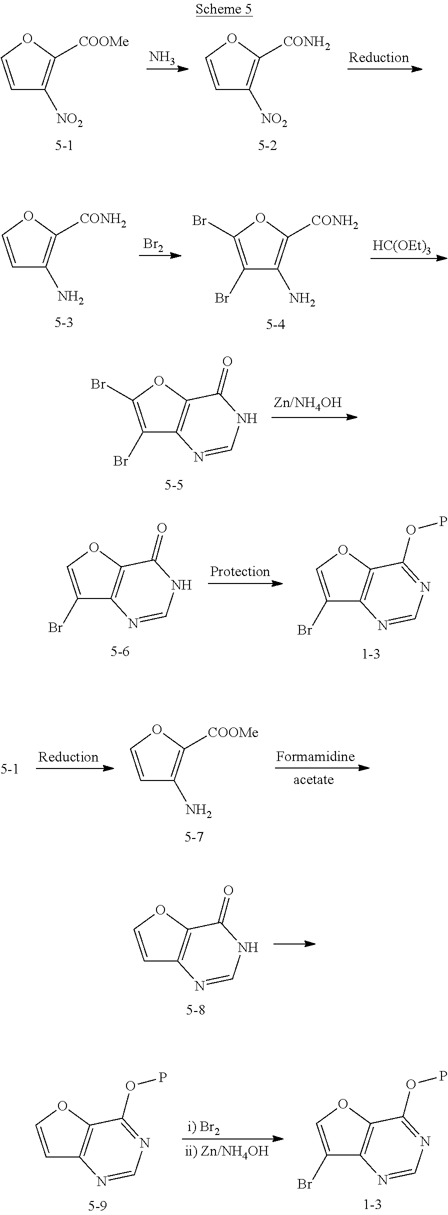

Preparation of Compound 1-3, when Y═H, X═Br, R═OP:

The starting material, 3-nitro-furan-2-carboxylic acid methyl ester 5-1, is prepared by the literature procedures (J. Org. Chem., 267-275 (2003)) and can be converted to compound 5-2 by treating with ammonia in a suitable solvent under appropriate conditions. The reduction of compound 5-2 generates compound 5-3, bromination yields a dibromo derivative, compound 5-4, and further cyclization of compound 5-4 with triethyl-orthoformate gives the required heterocycle furo[3,2-d]pyrimidine 5-5. Selective debromination of compound 5-5 is achieved through zinc/ammonium hydroxide or n-butyllithium exchange and the resultant compound 5-6 is produced which results in the desired compound 1-3 on protection.

Alternatively, compound 5-1 can be first reduced to amino to give compound 5-7, which upon cyclization with formamidine salt generates compound 5-8. Further manipulations are done the same way as given in Scheme 4 to yield compound 1-3.

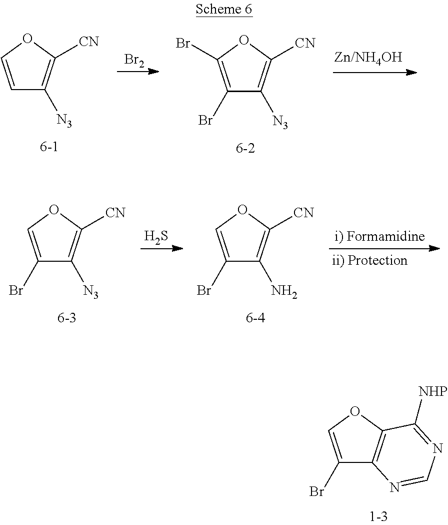

### Alternative Synthesis of Compound 1-3 is Described in Scheme 6:

The starting material, 3-azido-furan-2-carbonitrile 6-1, (Acta Chemia Scandinavia B 29, 224-232 (1975)) can be brominated to give dibromo compound 6-2, which is selectively debrominated at the 5-position using Zn/NH₄OH as described in J. Org. Chem. 2835-2846 (1976). The azido group of resultant compound 6-3 is reduced to amine 6-4 using H₂S similar to that described in Acta Chemia Scandinavia B 29, 224-232 (1975). Compound 6-4 is condensed with formamidine acetate and protected to give desired compound 1-3.

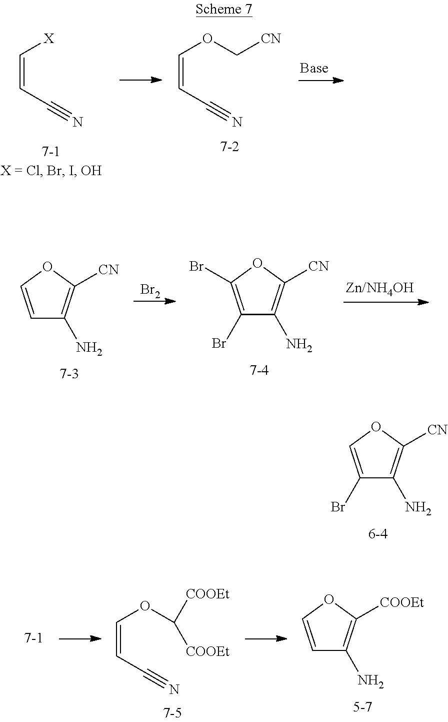

### Compounds 6-4 and 5-7 (Ethyl Ester Analog) can be Prepared as Given in Scheme 7:

The starting material 3-halo-acrylonitrile 7-1 (J. Org. Chem. 57, 708-713 (1992)) is treated with the sodium salt of 2-hydroxyacetonitrile to give compound 7-2 which is analogous to the reaction described in J. Med. Chem. 43, 4288-4312 (2000). 3-Hydroxypropenenitrile (J. Org. Chem. 56, 970-975 (1991)) on treatment with haloacetonitrile also generates 7-2. Compound 7-2 is treated with strong base, such as lithium-N,N-diisopropylamide or sodium ethoxide, to generate compound 7-3 (Tetrahedron Lett. 27, 815-818 (1986)), which upon bromination yields dibromo derivative 7-4. Selective debromination is obtained by the reaction with zinc and ammonium hydroxide to produce required compound 6-4.

Similarly, treatment of compound 7-1 with bromodiethylmalonate gives compound 7-5 which on base cyclization yields required compound 5-7.

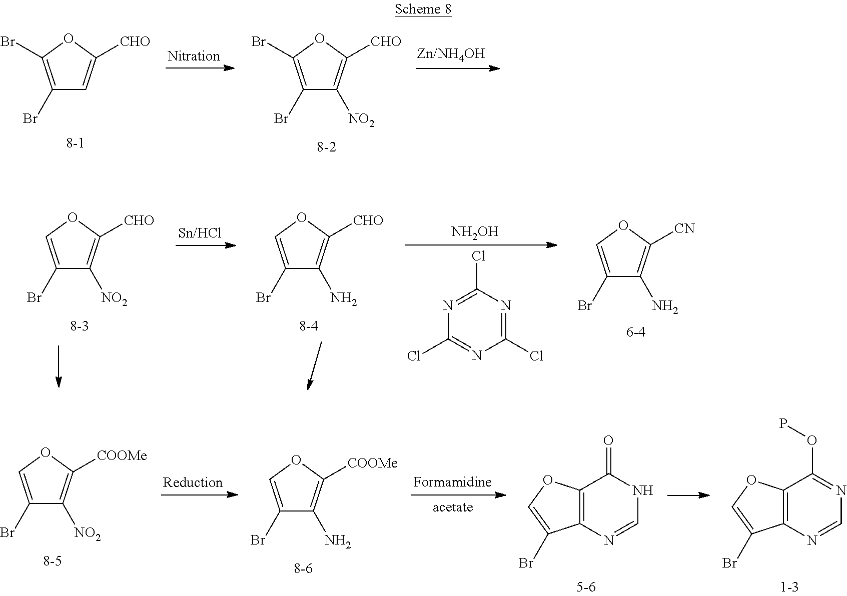

### Method for the Preparation of Compound 6-4, as Given in Scheme 8:

This scheme also describes the synthesis of compound 1-3 (when Y═H, X═Br, and R═OP) from the intermediates toward the preparation of compound 6-4. The starting material, 4,5-dibromo-furan-2-carbaldehyde 8-1 (Tetrahedron 44, 41-48 (1988)) is subjected to nitration to give compound 8-2, which upon selective debromination yields compound 8-3. The nitro group in compound 8-3 is reduced to amino generating compound 8-4 and the aldehyde group is converted to cyano producing compound 6-4 by treating with hydroxylamine followed by dehydration. Further conversions of compounds 8-3 and 8-4 also can result into the desired compound 1-3, as shown in Scheme 8.

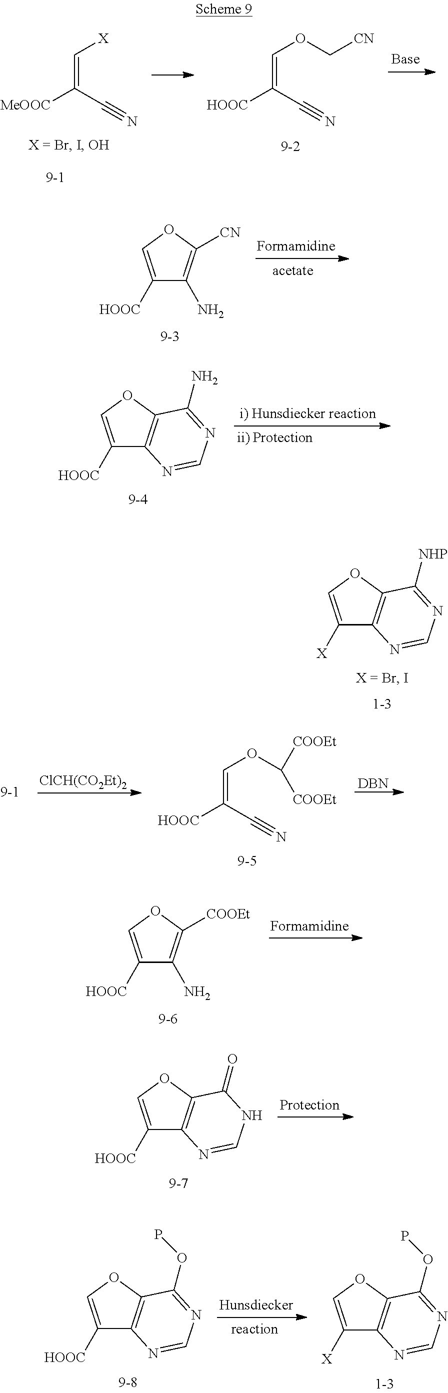

Method of Producing Compound 1-3 (when X═Br or I, Y═H, and R═NHP or OP) as Shown in Scheme 9 (Similar to Scheme 7):

The only difference with this method is that this starting material 9-1 (J. Org. Chem. 57, 6837-6842 (1992)) has one additional COOH present in the molecule group compared to compound 7-1, which, later in the synthesis, is transformed to bromo through the Hunsdiecker reaction.

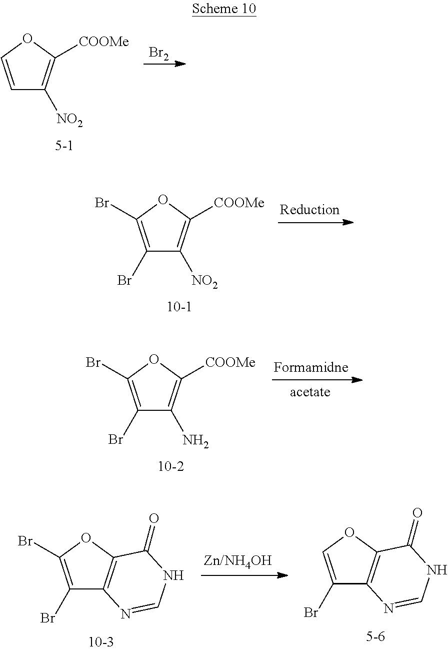

### Method for Preparation of Compound 5-6, as Described in Scheme 10:

Compound 5-1 is brominated to dibromo derivative 10-1 and the nitro group is reduced to amino using metal/acid reduction to generate compound 10-2. Usual cyclization of 10-2 with formamidine salt yields compound 10-3 and selective debromination with zinc and ammonium hydroxide gives the desired compound 5-6.

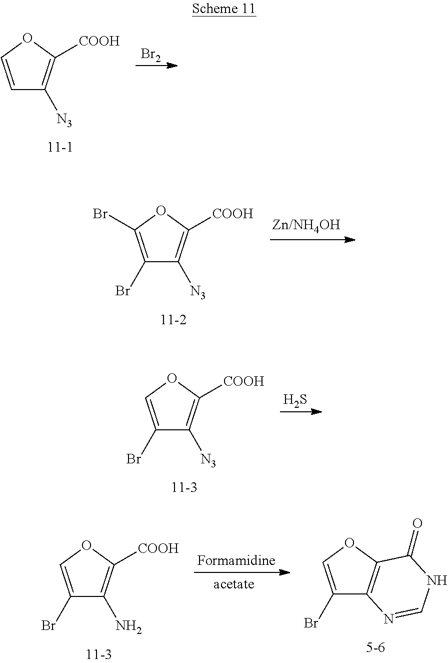

### Method for Preparation of Compound 10-4, as Described in Scheme 11:

3-Azido-furan-2-carboxylic acid 11-1 (Acta Chemia Scandinavia B 29, 224-232 (1975)) is brominated to give dibromo compound 11-2, which is selectively debrominated to give compound 11-3. The subsequent reduction of azide to amine with hydrogen sulfide gives compound 11-4 which is then cyclized to desired compound 5-6 using formamidine salt.

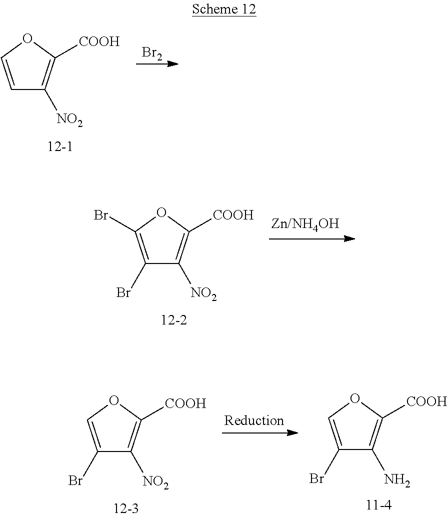

### Alternative Synthesis of 11-4, as Described in Scheme 12:

3-Nitro-furan-2-carboxylic acid 12-1 (Recl. Tray. Chim. Pays-Bass. 52, 390-392 (1938)) is brominated to give dibromo compound 12-2, which is selectively debrominated with zinc and ammonium hydroxide to give compound 12-3. The reduction of the nitro group to amino produces the desired compound 11-4.

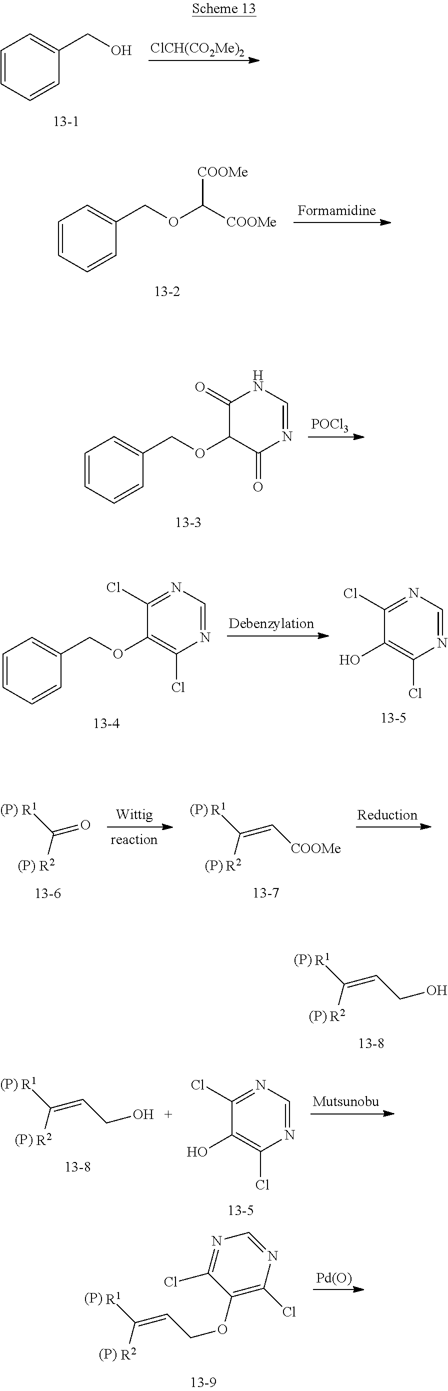

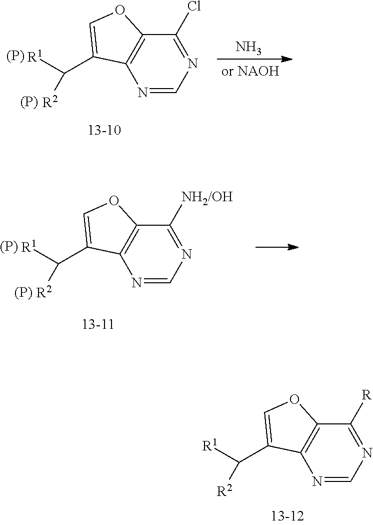

### Different Approach for the Preparation of Compound 1-7, as Shown in Scheme 13:

Benzyl alcohol 13-1 is alkylated with dimethylchloromalonate to give compound 13-2, which is condensed with formamidine salt and generates compound 13-3. Compound 13-3 on reaction with phosphorous oxychloride generates compound 13-4 which is then debenzylated to give compound 13-5.

In a separate reaction sequence, compound 13-7, obtained through Wittig olefination of compound 13-6, is reduced to alcohol 13-8 with standard reducing reagents.

The reaction of compound 13-8 with 13-5 under Mitsunobu conditions gives compound 13-9, which undergoes intramolecular cyclization to produce compound 13-10. The reaction conditions are described in Tetrahedron Lett. 44, 725-728 (2003). The chloro group is converted to amino or hydroxy and the resultant compound 13-11 on deprotection affords the target molecule 13-12.

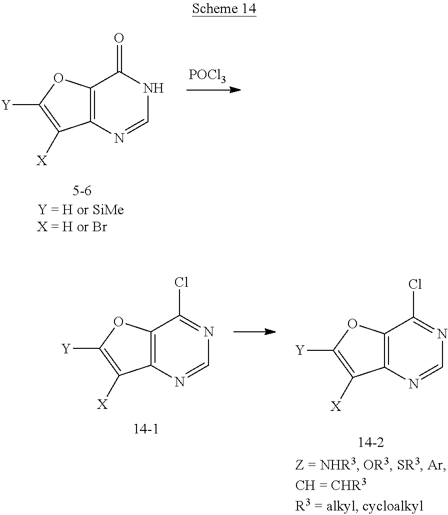

Compounds with Other Substituents at 4-Position are Obtained as Shown in Scheme 14:

Compound 5-6 is converted to compound 14-1 with POCl₃ and chloro is substituted with substituted amines to give Z═NHR³; with alkoxides to give Z═OR³; with thio-alkoxides to give Z═SR³, with aryl boronic acids to give Z═Ar; and with alkene boronic acids to give Z═CH═CHR³.

The following documents illustrate synthetic procedures that can be used to carry out the reactions described in the above schemes.

### General Conversions

### Conversion of Halide to Azide (Example, 4-1 to 4-2):

This conversion can be achieved by reacting halide with the reagents, like lithium azide, sodium azide, trimethylsilyl azide, hydrazoic acid in solvents such as, dimethyl sulfoxide, dimethyl formamide, tetrahydrofuran, dimethyl ethyleneglycol etc., at room temperature to elevated temperatures.
1. Acta Chemia Scandinavia B 29, 224-232 (1975).

### Conversion of Azide to Amine (Examples, 4-2 to 4-3, 6-3 to 6-4, 11-3 to 11-4):

The azide to amine reduction can be achieved by several different reagents and conditions such as, catalytic hydrogenation using palladium or platinum in solvents like methanol or ethanol; hydride reduction with diborane, lithium dimethylamino borohydride, sodium borohydride, zinc borohydride, lithium aluminum hydride, n-butyltin hydride; metal reduction with magnesium in methanol, calcium in methanol, zinc and HCl, zinc and acetic acid; with hydrogen sulfide in water and pyridine; or triphenyl phosphine and water.
1. Acta Chemia Scandinavia B 29, 224-232 (1975).
2. Chem Rev. 54, 1 (1954).
3. J. Med Chem. 12, 658 (1969).
4. J. Am. Chem. Soc. 87, 4203 (1965).
5. J. Org. Chem. 59, 6378 (1994).
6. J. Am. Chem. Soc. 73, 5865 (1951).
7. Syn. Commun. 18, 1201 (1988).
8. J. Org. Chem. 54, 3292 (1989).
9. J. Org. Chem. 59, 7944 (1994).
10. J. Org. Chem. 52, 5044 (1987).
   Formation of Pyrimidine Ring Attached with Furan (Examples, 4-3 to 4-4, 5-4 to 5-5, 5-7 to 5-8, 6-4 to 1-3, 8-6 to 5-6, 9-3 to 9-4, 9-6 to 9-7, 10-2 to 10-3, 11-4 to 5-6):

Furo[3,2-d]pyrimidin-4-one derivatives can be synthesized from furan substituted with amine and adjacent ester or acid by reacting with formamidine salt. When there is amide in place of ester, the reaction with triethyl orthoformate, acetic acid, acetic anhydride, 2,2,2-trifluoro-acetamide or dimethylformamide affords the desired compound.

Furo[3,2-d]pyrimidin-4-one derivatives can be synthesized from furan substituted with amine and adjacent nitrile by reacting with formamidine salt in ethanol or 2-methoxyethanol at temperatures from 20° C. to 100° C. the reaction with triethyl orthoformate and ammonia in dimethylformamide and ethanol at reflux temperature also affords the desired compound.
1. Tetrahedron Lett. 22 (44), 4397-4400 (1981).
2. J. Org. Chem. 17, 149-153 (1952).
3. J. Chem. Soc. Perkin Trans. 1, 2304-2309 (1980).
4. Bull. Soc. Chim. Fr., 587-591 (1975).
5. J. Chem. Soc, 2329-2331 (1949).
6. J. Med. Chem. 32, 1757-1763 (1989).
7. J. Chem. Soc. Perkin. Trans. 1 (18), 2259-2264 (1992).
8. Bioorg. Med. Chem. Lett. 10, 2223-2226 (2000).
9. J. Heterocycl. Chem. 12, 111-117 (1975).
10. Eur. J. Med. Chem. Chim. Ther. 11, 67-72 (1976).
11. Synthesis 2, 157-159 (1991).
12. Heterocycles 55, 2279-2282 (2001).
13. J. Med. Chem. 48(23), 7445-56 (2005).

### Bromination (Examples, 4-5 to 1-3, 4-8 to 1-3, 5-3 to 5-4, 5-9 to 1-3, 6-1 to 6-2, 7-3 to 7-4, 5-1 to 10-1, 11-1 to 11-2, 12-1 to 12-2):

Bromination can be achieved by treatment of the aromatic compound with bromine in acetic acid or with N-bromosuccinimide in the presence of HClO₄ or sulfuric acid.
1. J. Org. Chem. 52, 60 (1987).
2. J. Org. Chem. 58, 3072 (1993).
3. J. Org. Chem. 30, 304 (1965).
4. Tetrahedron 44, 1-41 (1988).

### Conversion of Hydroxyl or Oxo Derivative to Chloro (Examples, 4-4 to 4-6, 13-3 to 13-4, 5-6 to 14-1):

This transformation can be accomplished by treatment with, phosphorous oxychloride in solvents such as, acetonitrile in the presence of N,N-dimethylaniline, pyridine, benzenetriethylammonium chloride, or triethylammonium chloride; thionyl chloride in the presence of N,N-diethylaniline; or triphenyl phosphine, carbontetrachloride and diazabicyclo-5,4-undecene in solvents like chloroform or dichloromethane.
1. Org. Lett. 6, 2917-2919 (2004).
2. J. Org. Chem. 66, 5723-5730 (2001).
3. J. Med Chem. 47, 1339-1350 (2004).
4. J. Med Chem. 26, 1601-1606 (1983).
5. J. Chem. Soc. Perkin Trans. 1 8, 923-926 (1994).

### Conversion of Ester to Amide (Example, 5-1 to 5-2):

Ester to amide transformation can be achieved by treatment of ester with methanolic or ethanolic ammonia solutions; sodium amide in ammonia; or ammonium hydroxide in the presence of ammonium chloride.
1. Organic synthesis collective, 1, 153-179 (1941).
2. Organic synthesis collective, 3, 516-536 (1955).
3. Organic synthesis collective, 4, 486 (1963).
4. J. Org. Chem. 52, 437 (1987).
5. J. Org. Chem. 56, 146-151 (1991).
6. J. Org. Chem. 56, 4499 (1991).

### Reduction of Nitro Group to Amine (Examples, 5-2 to 5-3, 5-1 to 5-7, 8-3 to 8-4, 8-5 to 8-6, 10-1 to 10-2, 12-3 to 11-4):

Reduction of the aromatic nitro group can be achieved by several reagents such as, catalytic hydrogenation using Raney Ni, platinum oxide, or palladium on carbon; hydrazine and FeCl₃, Fe(III) oxide or palladium on carbon; acid with different metals like tin, iron, or zinc; sulfide reagents like sodium sulfide or ammonium sulfide; hydride reagents like sodium tellurium hydride or sodium borohydride with different metals such as palladium, tin, titanium, or nickel.
1. J. Org. Chem. 18, 1506 (1953).
2. J. Org. Chem. 60, 1939 (1995).
3. Organic synthesis collective, 3, 59-63 (1955).
4. Organic synthesis collective, 5, 829 (1973).
5. Organic synthesis collective, 1, 240 (1941).
6. Chem. Rev. 65, 51 (1965).
7. J. Org. Chem. 59, 192 (1994).
8. Synthesis, 834 (1978).
9. Tetrahedron Lett. 23, 147 (1982).
10. Organic synthesis collective vol. 5, 30 (1973).
11. Organic synthesis collective vol. 1, 455 (1941).
12. J. Org. Chem. 60, 4006 (1995).
13. J. Org. Chem. 60, 3365 (1995).
14. Proc. Ind. Acad. Sci. A 44, 331 (1956).
15. Organic synthesis collective vol. 5, 1067 (1973).
16. Chemistry Lett. 1373 (1983).
17. J. Org. Chem. 60, 4006 (1995).
18. Synthesis, 695 (1980).

### Selective Debromination (Examples, 5-5 to 5-6, 5-9 to 1-3, 6-2 to 6-3, 7-4 to 6-4, 8-2 to 8-3, 10-3 to 5-6, 11-2 to 11-3, 12-2 to 12-3):

Selective debromination can be achieved by treatment of the dibromo compound with reagents like zinc or n-butyl lithium and quenching with a proton donor like ammonium hydroxide.
1. Tetrahedron 44, 1-41 (1988).
2. J. Org. Chem. 2835-2846 (1976).

### Furan Ring Synthesis (Examples, 7-2 to 7-3, 7-5 to 5-7, 9-2 to 9-3, 9-5 to 9-6):

Furan ring synthesis can be accomplished by treatment of a compound like 7-2 with a strong base such as lithium N,N-diisopropylamide or sodium ethoxide or treatment of compounds similar to 7-5 with DBN.
1. Tetrahedron Lett. 27, 815-818 (1986).

### Nitration (Example, 8-1 to 8-2):

Nitration of an aromatic compound can be achieved by treatment with sulfuric acid and nitric acid or nitric acid at lower temperatures.
1. Organic synthesis coll., 3, 837 (1955).
2. Organic syntheses, coll., 5, 480 (1973); vol. 47, 56 (1967).

### Aldehyde to Nitrile Transformation (Example, 8-4 to 6-4):

Aldehydes can be transformed to corresponding aldoximes by treatment of hydroxylamine and then dehydrated to nitrile by using 2,4,6-trichloro-s-triazine.
1. Acta Chemia Scandinavia B 29, 224-232 (1975).
2. J. Org. Chem. 17, 6272-6274 (2002).
3. J. Am. Chem. Soc. 29, 10124 (2005).

### Conversion of Acid to Halo, Hunsdiecker Reaction (Examples, 9-4 to 1-3, 9-8 to 1-3):

This conversion can be achieved by treatment of anhydrous silver salts of an organic acid with bromine or iodine in solvents like carbon tetrachloride. Alternatively, organic acid can be treated with sodium hydroxide solution followed by potassium iodide.
1. U.S. Pat. No. 2,176,181 (1939).
2. Organic Reactions 9, 341 (1957).
3. J. Org. Chem. 28, 48 (1963).
4. J. Am. Chem. Soc. 54, 733-735 (1932).
5. Synthesis 1319-1325 (2005).

### Alkylation (Example, 13-6 to 13-7):

Compound 13-2 can be prepared by treatment of benzyl alcohol with sodium methoxide and dimethyl chloromalonate in solvents like methanol.
1. Bioorg. Med. Chem. 9, 897-907 (2000).

### Wittig Reaction (13-6 to 13-7):

Compound 13-7 can be prepared by treatment of a ylide from a corresponding phosphonate with a ketone in solvents like THF, DMF, etc.
1. Org. React. 25, 73-253 (1977).
2. Acc. Chem. Res. 16, 411-417 (1983).
3. Chem. Rev. 74, 87-99 (1974).

### Ester to Alcohol (Example, 13-7 to 13-8):

Esters can be reduced to alcohols with reducing agents like diisobutyl aluminum hydride or lithium aluminum hydride in solvents like diethyl ether.
1. Tetrahedron Lett. 32, 1649-1652 (1991).
2. Tetrahedron Lett. 1287-1290 (1972).
3. J. Org. Chem. 49, 3859-3860 (1984).

### Mitsunobu Reaction (Example, 13-8 to 13-9):

Allyl alcohols can be treated with triphenylphosphine and DEAD followed by an aromatic alcohol in solvents like tetrahydrofuran to give ether compound 13-9.
1. Tetrahedron Lett. 44, 725-728 (2003).
   Cyclization with Pd (0) (Example, 13-9 to 13-10):

This transformation can be achieved from palladium acetate in buffer of sodium carbonate, sodium formate, and tetrabutylammonium chloride using DMF as solvent at room temperature to 100° C.
1. Tetrahedron Lett. 44, 725-728 (2003).

In compounds of formula (I), (IV), and (V) the group R² is a nucleoside sugar group. The values for R² include any group that can function as a nucleoside sugar group in the compound. Numerous examples of nucleoside sugar groups are illustrated in the following groups A-F. The values for R², however, are not limited to those values illustrated herein.

### Group A

Examples of substituted tetrahydro and dihydrofuranyl compounds include those compounds represented by the general structures:

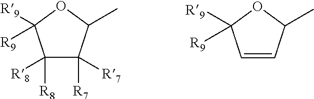

Specific examples include, but are not limited to, the following compounds:

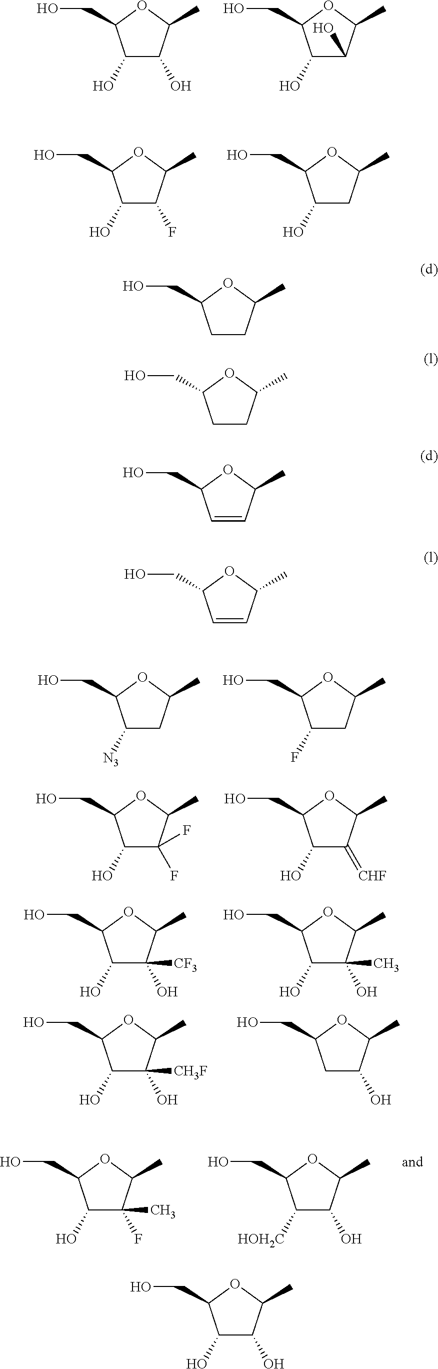

### Group B

Examples of substituted tetrahydrothiophenyl and dihydrothiophenyl compounds include those compounds represented by the general structures:

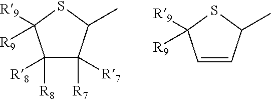

Specific examples include, but are not limited to, the following compounds:

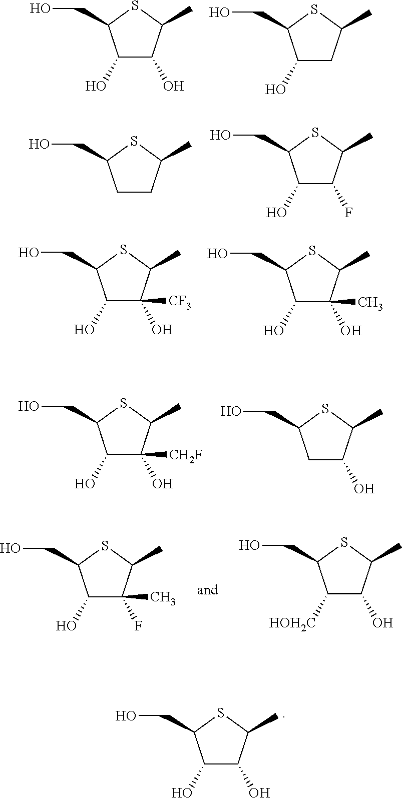

### Group C

Examples of substituted alkyl compounds include those compounds represented by:

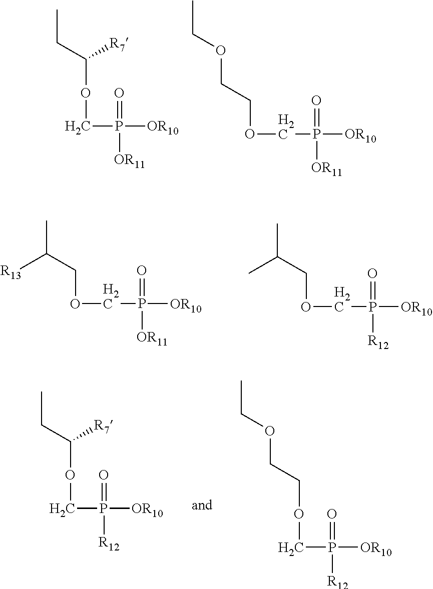

Specific examples include, but are not limited to, the following compounds:

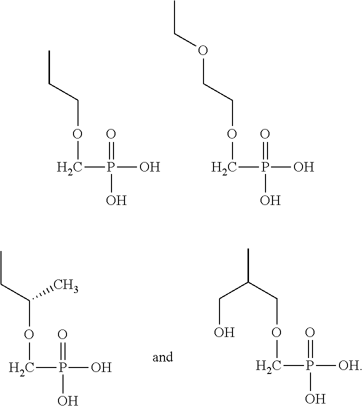

### Group D

Examples of substituted cycloalkyl and cycloalkenyl compounds include those compounds represented by the general structures:

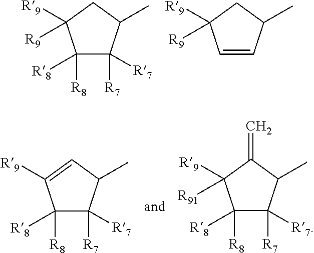

Specific examples include, but are not limited to, the following compounds:

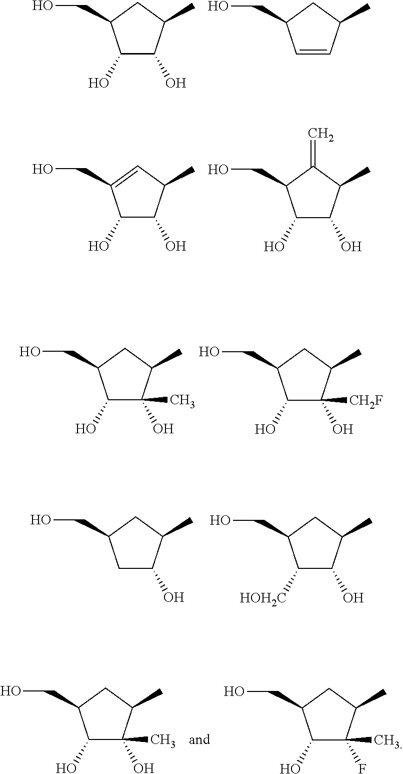

### Group E

Examples of substituted dihydropyrrolidinyl and tetrahydropyrrolidinyl compounds include those compounds represented by the general structures:

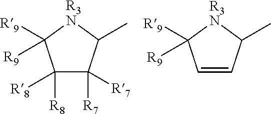

Specific examples include, but are not limited to, the following compounds:

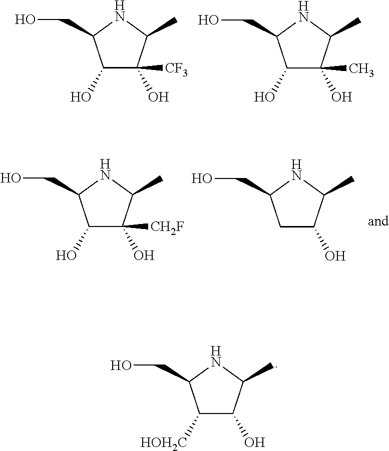

### Group F

Examples of substituted dioxolane, substituted thioxolane and substituted dithiolane compounds include those compounds represented by the general structures:

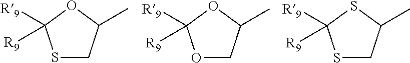

Specific examples include, but are not limited to, the following compounds:

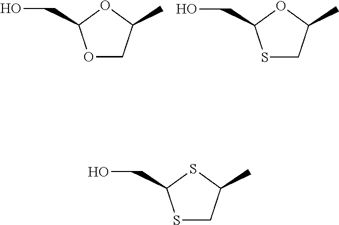

For the structures in Groups A-F, the following definitions apply:

R₇ is H, OR₁₄, N₃, NH₂, or F; and R′₇ is H, F, OH, O-alkyl, alkyl, substituted alkyl, alkenyl, substituted alkenyl, alkynyl, or substituted alkynyl; or R₇ and R′₇ together may be ═CH₂, ═CHF; wherein both R₇ and R′₇ are not OH; and when one of R₇ and R′₇ is NH₂, the other is not OH; and when one of R₇ and R′₇ is N₃, the other is not OH;

R₈ is H, OR₁₄, N₃, NH₂, or F; and R′₈ is H, F, OH, O alkyl, alkyl, substituted alkyl, alkenyl, substituted alkenyl, alkynyl, or substituted alkynyl; or R₈ and R′₈ together may be ═CH₂, ═CHF; wherein both R₈ and R′₈ are not OH; and when one of R₈ and R′₈ is NH₂, the other is not OH; and when one of R₈ and R′₈ is N₃, the other is not OH;

R₉ is H, CH₃, C₂H₅, or N₃;

R′₉ is CH₂OR₁₄, CH₂F, CH₂SH, CHFOH, CF₂OH,

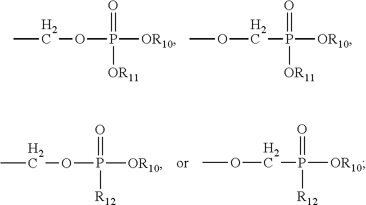

R₁₀ and R₁₁ are each independently H, alkyl, aryl, substituted aryl, acyloxyalkyl, or (CH₂)ₙ—O—(CH₂)ₘCH₃;

R₁₂ is an N-linked amino acid residue (e.g. —NH—CH(CH₃)CO₂alkyl or —NH—CH(isopropyl)-CO₂alkyl);

R₁₃ is H, CH₃, C₂H₅, CH₂F, CH₂OH, CH₂CH₂F, CH₂CH₂OH, CH₂N₃, CH₂CH₂N₃, CH₂NH₂, or CH₂CH₂NH₂; and

R₁₄ is H.

In one embodiment, R₁₄ is replaced to form a pharmaceutically acceptable prodrug, for example, a prodrug selected from the group consisting of: acyl, oxyacyl, phosphonate, phosphate, phosphate esters, phosphonamidate, phosphorodiamidate, phosphoramidate mono ester, cyclic phosphoramidate, cyclic phosphorodiamidate, phosphoramidate diester, C(O)CHR₁₅NH₂.

Synthetic processes and intermediates that can be used to prepare nucleoside sugar groups are described in the following sources.
1. Can. J. Chem. 36, 1720, 1725 (1958).
2. Org. Lett. 5, 4277-4280 (2003).
3. J. Am. Chem. Soc. 124, 7061-7069 (2002).
4. J. Bacteriol. 74, 180, 182 (1957).
5. J. Am. Chem. Soc. 47, 3022 (1925).
6. Tetrahedron Lett. 37, 2237-2240 (1996).
7. J. Chem. Commun. 5, 445-446 (1992).
8. J. Org. Chem. 64, 4238-4246 (1999).
9. Acta Chem. Scand. Ser. B 38, 689-694 (1984).
10. Org. Lett. 6, 3949-3952 (2004).
11. Org. Lett. 7, 1813-1816 (2005).
12. J. Am. Chem. Soc. 115, 3558-3575 (1993).
13. Tetrahedron 60, 7621-7628 (2004).
14. J. Am. Chem. Soc. 114, 1059-1070 (1992).
15. J. Org. Chem. 69, 7822-7829 (2004).
16. Carbohydr. Res. 216, 511-516 (1991).
17. Synthesis 10, 1291-1294 (1995).
18. Tetrahedron: Asymmetry 11, 1869-1876 (2000).
19. Tetrahedron Lett. 29, 5349-5352 (1988).
20. J. Org. Chem. 54, 5171-5176 (1989).
21. Carbohydr. Res. 306, 69-80 (1998).
22. Tetrahedron 54, 14023-14030 (1998).
23. Carbohydr. Chem. 13, 767-776 (1994).
24. J. Chem. Soc. 2301, 2304 (1951).
25. Bull. Chem. Soc. Jpn. 61, 2025-2030 (1988).
26. Tetrahedron Lett. 31, 6931-6934 (1990).
27. J. Org. Chem. 50, 4442-4447 (1985).
28. J. Org. Chem. 58, 7860-7864 (1993).
29. Tetrahedron Lett. 36, 1941-1944 (1995).
30. J. Chem. Soc. Chem. Commun. 10, 740-741 (1991).
31. Tetrahedron 58, 7075-7080 (2002).
32. Tetrahedron 24, 5559-5562 (1983).
33. J. Am. Chem. Soc. 117, 5391-5392 (1995).
34. J. Org. Chem. 66, 8831-8842 (2001).
35. Chem. Pharm. Bull. 35, 2140-2143 (1987).

### Specific Embodiments

In one specific embodiment the invention provides a method for preparing a compound of formula IV:

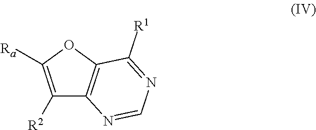

wherein:

R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;

R² is a nucleoside sugar group;

n is 0-5;

R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;

Rₐ is H; or a protected analog thereof;

comprising: reacting a corresponding compound of formula III:

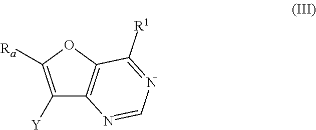

wherein Y is a metallic group, with an electrophilic nucleoside sugar group precurser; to provide the compound of formula (IV).

In one specific embodiment the invention provides a method for preparing a compound of formula IV:

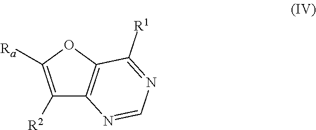

wherein:

R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;

R² is a nucleoside sugar group;

n is 0-5;

R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;

Rₐ is H; or a protected analog thereof;

comprising: deoxygenating a corresponding compound of formula (V)

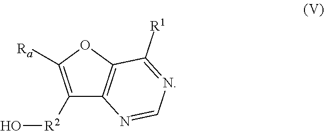

In one specific embodiment the invention provides a method for preparing a compound of formula (V) by reacting a corresponding compound of formula III:

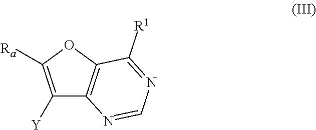

wherein: Y is a metallic group; or a protected analog thereof;
with an electrophilic nucleoside sugar group precurser of formula R²(═O),
wherein R² is a nucleoside sugar group.

In one specific embodiment the invention provides a method for preparing a compound of formula (V) by reacting a corresponding compound of formula III:

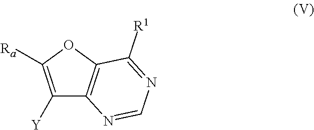

wherein: Y is a metallic group; or a protected analog thereof;
with an electrophilic nucleoside sugar group epoxide precurser of formula R²(>O), wherein R² is a nucleoside sugar group.

In one specific embodiment the invention provides a compound of formula (V):

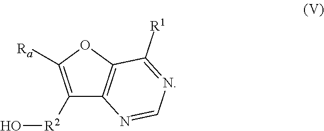

wherein:

R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;

n is 0-5;

R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;

Rₐ is H; and

R² is a nucleoside sugar group.

In one specific embodiment the invention provides a method for preparing a compound of formula IV:

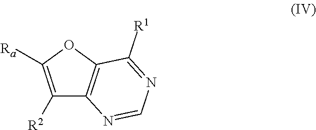

wherein:

R¹ is OR_{b} or NHR_{c};

R_{b} is a hydroxy protecting group;

R_{c} is an amino protecting group;

Rₐ is H; and

R² is a nucleoside sugar group;

comprising: reacting a corresponding compound of formula III:

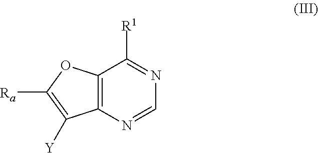

wherein Y is a metallic group, with an electrophilic nucleoside sugar group precursor; to provide the compound of formula (IV).

In one specific embodiment the invention provides a method for preparing a compound of formula IV:

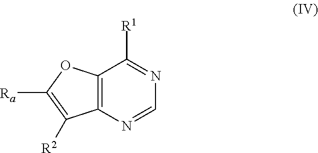

wherein:

R¹ is OR_{b} or NHR_{c};

R_{b} is a hydroxy protecting group;

R_{c} is an amino protecting group;

Rₐ is H; and

R² is a nucleoside sugar group;

comprising: deoxygenating a corresponding compound of formula (V)

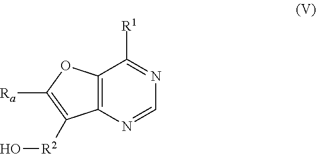

In one specific embodiment the invention provides a method for preparing a compound of formula (V) by reacting a corresponding compound of formula III:

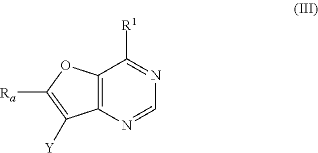

wherein:

Y is a metallic group;

with an electrophilic nucleoside sugar group precursor of formula R²(═O),
wherein R² is a nucleoside sugar group.

In one specific embodiment the invention provides a method for preparing a compound of formula (V) by reacting a corresponding compound of formula III:

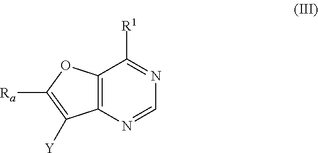

wherein:

Y is a metallic group;

with an electrophilic nucleoside sugar group epoxide precursor of formula R²(>O), wherein R² is a nucleoside sugar group.

In one specific embodiment the invention provides a compound of formula (V):

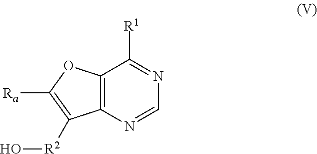

wherein:

R¹ is OR_{b} or NHR_{c};

R_{b} is a hydroxy protecting group;

R_{c} is an amino protecting group;

Rₐ is H; and

R² is a nucleoside sugar group.

In one specific embodiment the invention provides a method for preparing a compound of the following formula (a):

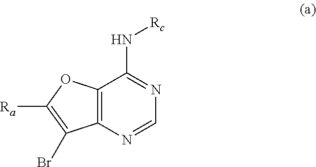

wherein Rₐ is H or Si(CH₃)₃, and R_{c} is an amino protecting group; comprising brominating a corresponding compound of formula (b):

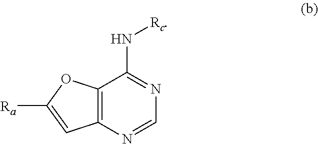

In one specific embodiment the invention provides a method for preparing a compound of formula (b) by protecting a corresponding amino compound of formula (c):

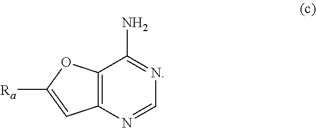

In one specific embodiment the invention provides a method for preparing a compound of formula (c) by converting a corresponding chloride of formula (d):

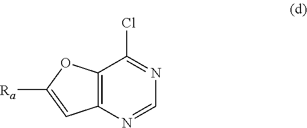

to the amino compound of formula (c).

In one specific embodiment the invention provides a method for preparing a chloro compound of formula (d) by chlorinating a corresponding compound of formula (e):

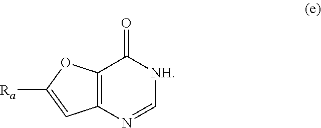

In one specific embodiment the invention provides a method for preparing a compound of formula (e) by cyclizing a corresponding compound of formula (f):

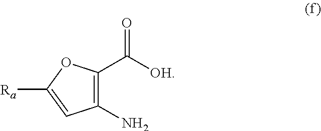

In one specific embodiment the invention provides a method for preparing a compound of formula (f) by reducing a corresponding azide of formula (g):

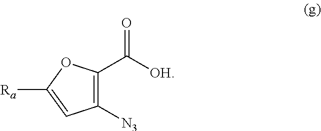

In one specific embodiment the invention provides a method for preparing an azide of formula (g) by converting a corresponding iodide of formula (h):

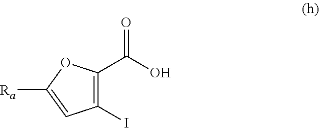

to the azide.

In one specific embodiment the invention provides a method for preparing a compound of the following formula (i):

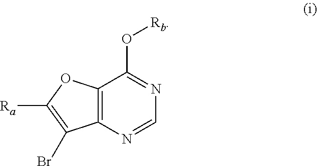

wherein Rₐ is H or Si(CH₃)₃, and R_{b} is a hydroxy protecting group; comprising brominating a corresponding compound of formula (j):

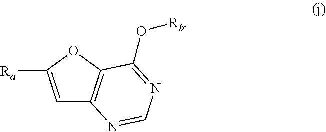

In one specific embodiment the invention provides a method for preparing a compound of formula (j) by protecting a corresponding compound of formula (e):

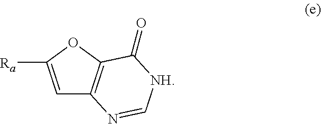

In one specific embodiment the invention provides a method for preparing a method for preparing a compound of the following formula (k):

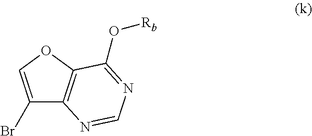

wherein R_{b} is a hydroxy protecting group comprising protecting a corresponding compound of formula (l):

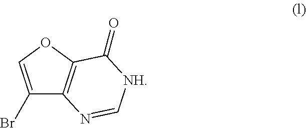

In one specific embodiment the invention provides a method for preparing a compound of formula (l) by reducing a corresponding di-bromide of formula (m):

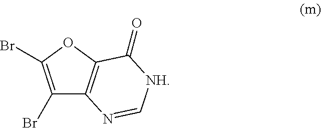

In one specific embodiment the invention provides a method for preparing a compound of formula (m) by cyclizing a corresponding compound of formula (n):

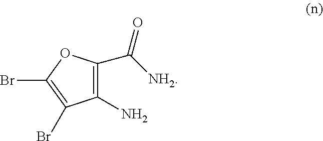

In one specific embodiment the invention provides a method for preparing a compound of formula (n) by brominating a corresponding compound of formula (az):

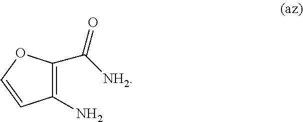

In one specific embodiment the invention provides a method for preparing a compound of formula (az) by reducing a corresponding nitro compound of formula (o):

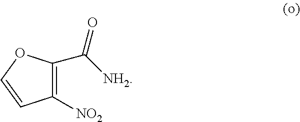

In one specific embodiment the invention provides a method for preparing a compound of formula (o) by converting a corresponding ester of formula (p):

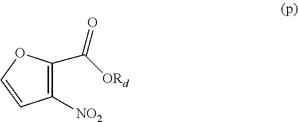

wherein R_{d} is alkyl to the compound of formula (p).

In one specific embodiment the invention provides a method for preparing a compound of the following formula (k):

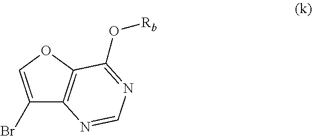

wherein R_{b} is a hydroxy protecting group comprising brominating a corresponding compound of formula (q):

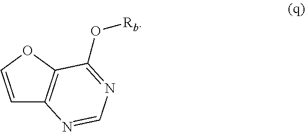

In one specific embodiment the invention provides a method for preparing a compound of formula (q) by protecting a corresponding compound of formula (r):

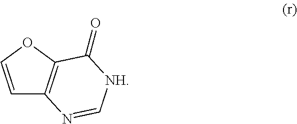

In one specific embodiment the invention provides a method for preparing a compound of formula (r) by cyclizing a corresponding compound of formula (s):

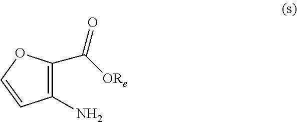

wherein Rₑ is alkyl.

In one specific embodiment the invention provides a method for preparing a compound of the following formula (a):

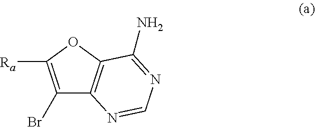

wherein Rₐ is H; comprising cyclizing a corresponding compound of formula (t):

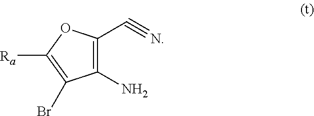

In one specific embodiment the invention provides a method for preparing a compound of formula (t) by reducing a corresponding azide of formula (u):

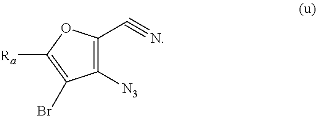

In one specific embodiment the invention provides a method for preparing a compound of formula (u) by reducing a corresponding dibromide of formula (v):

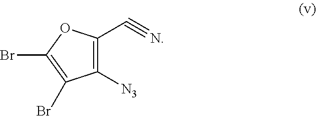

In one specific embodiment the invention provides a method for preparing a compound of formula (v) by brominating a corresponding compound of formula (w):

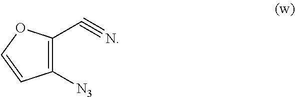

In one specific embodiment the invention provides a method for preparing a compound of formula (s):

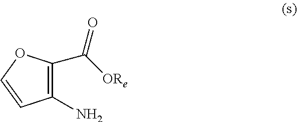

wherein Rₑ is alkyl comprising cyclizing a corresponding compound of formula (x):

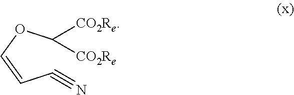

In one specific embodiment the invention provides a method for preparing a compound of formula (x) by alkylating a corresponding compound of formula (y):

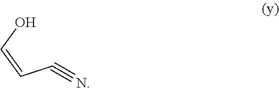

In one specific embodiment the invention provides a method for preparing a compound of formula (t):

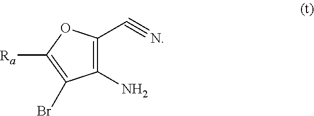

wherein R^{a} is hydrogen comprising reducing a corresponding dibromide of formula (z):

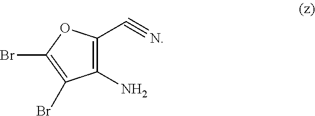

In one specific embodiment the invention provides a method for preparing a compound of formula (z) by brominating a corresponding compound of formula (aa):

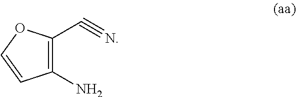

In one specific embodiment the invention provides a method for preparing a compound of formula (aa) by cyclizing a corresponding compound of formula (ab):

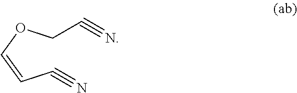

In one specific embodiment the invention provides a method for preparing a compound of formula (ab) by alkylating a corresponding compound of formula (y):

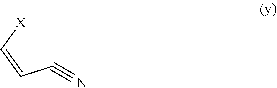

wherein X is Cl, Br, I, or OH.

In one specific embodiment the invention provides a method for preparing a compound of formula (t):

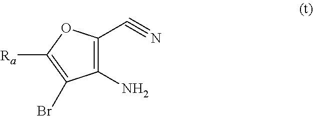

wherein Rₐ is hydrogen comprising converting a corresponding aldehyde of formula (ac):

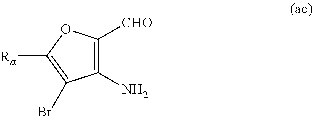

to the nitrile of formula (t).

In one specific embodiment the invention provides a method for preparing an aldehyde of formula (ac) by reducing a corresponding nitro compound of formula (ad):

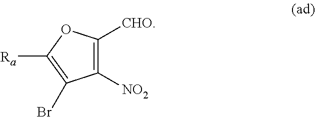

In one specific embodiment the invention provides a method for preparing a nitro compound of formula (ad) by reducing a corresponding dibromide of formula (ae):

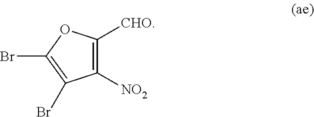

In one specific embodiment the invention provides a method for preparing a di-bromo compound of formula (ae) by nitrating a corresponding compound formula (af):

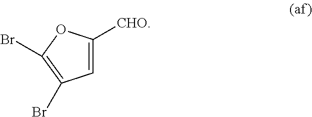

In one specific embodiment the invention provides a method for preparing a compound of formula (l):

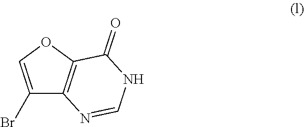

comprising cyclizing a corresponding compound of formula (ag):

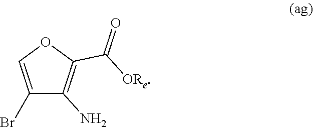

wherein Rₑ is alkyl.

In one specific embodiment the invention provides a method for preparing a compound of formula (ag) by converting an aldehyde of formula (ac):

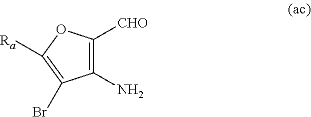

wherein Rₐ is hydrogen to the compound of formula (ag).

In one specific embodiment the invention provides a method for preparing a compound of formula (ag) by reducing a corresponding nitro compound of formula (ah):

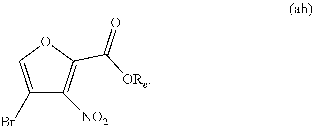

In one specific embodiment the invention provides a method for preparing a compound of formula (ah) by converting a corresponding aldehyde of formula (ad):

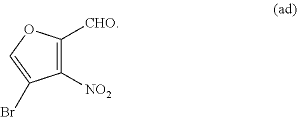

In one specific embodiment the invention provides a method for preparing a compound of the following formula (a):

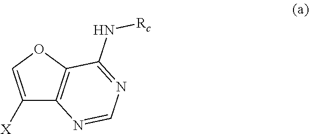

wherein R_{c} is an amino protecting group and X is Br or I; comprising protecting a corresponding compound of formula (ai):

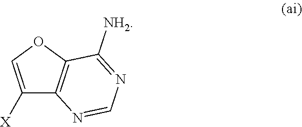

In one specific embodiment the invention provides a method for preparing a compound of formula (ai) by converting a corresponding acid of formula (aj):

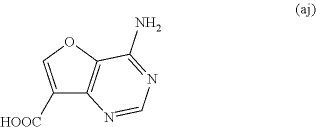

to the compound of formula (ai).

In one specific embodiment the invention provides a method for preparing a compound of formula (aj) by cyclizing a corresponding compound of formula (ak):

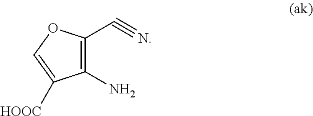

In one specific embodiment the invention provides a method for preparing a compound of formula (ak) by cyclizing a corresponding compound of formula (al):

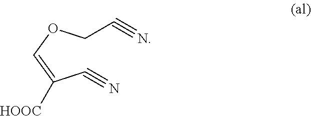

In one specific embodiment the invention provides a method for preparing a compound of formula (al) by converting a corresponding compound of formula (am):

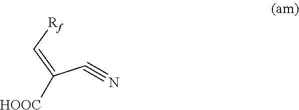

wherein R_{f} is Br, OH, or I to the compound of formula (al).

In one specific embodiment the invention provides a method for preparing a compound of the following formula (i):

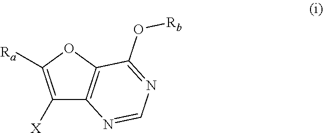

wherein Rₐ is H; X is Br or I; and R_{b} is a hydroxy protecting group; comprising converting a corresponding acid of formula (an) to the compound of formula (i).

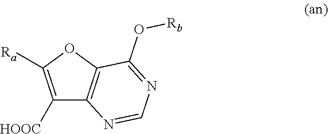

In one specific embodiment the invention provides a method for preparing an acid of formula (an) by protecting a corresponding compound of formula (ao):

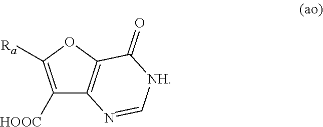

In one specific embodiment the invention provides a method for preparing a compound of formula (ao) by cyclizing a corresponding compound of formula (ap):

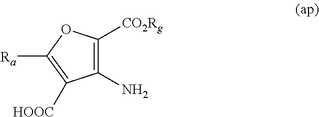

wherein R_{g} is alkyl.

In one specific embodiment the invention provides a method for preparing a compound of formula (ap) by cyclizing a corresponding compound of formula (aq):

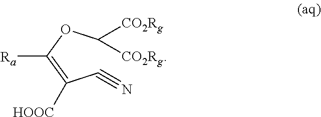

In one specific embodiment the invention provides a method for preparing a compound of formula (aq) by alkylating a corresponding compound of formula (am):

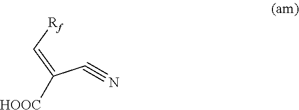

wherein R_{f} is OH.

In one specific embodiment the invention provides a method for preparing a compound of formula (l):

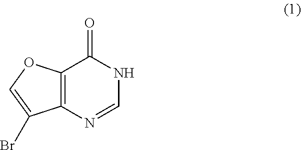

comprising reducing a corresponding di-bromide of formula (ar):

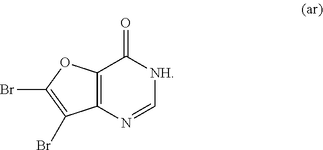

In one specific embodiment the invention provides a method for preparing a di-bromide of formula (ar) by cyclizing a corresponding compound of formula (as):

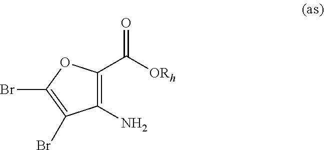

wherein Rₕ is alkyl.

In one specific embodiment the invention provides a method for preparing a compound of formula (as) by reducing a corresponding nitro compound of formula (at):

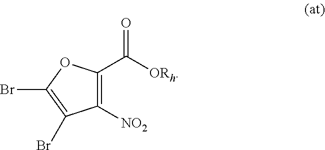

In one specific embodiment the invention provides a method for preparing a nitro compound of formula (at) by brominating a corresponding compound of formula (au):

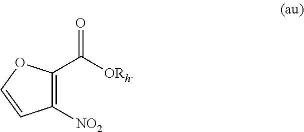

In one specific embodiment the invention provides a method for preparing a compound of formula (l):

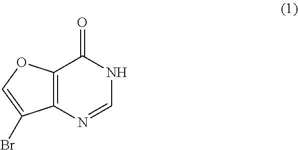

comprising cyclizing a corresponding compound of formula (av):

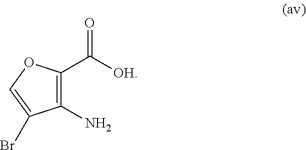

In one specific embodiment the invention provides a method for preparing a preparing the compound of formula (av) by reducing a corresponding azido compound of formula (aw):

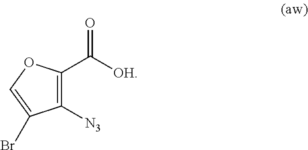

In one specific embodiment the invention provides a method for preparing an azido compound of formula (aw) by reducing a di-bromide of formula (ax):

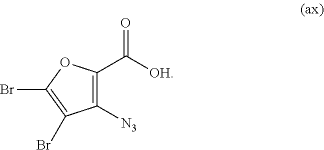

In one specific embodiment the invention provides a method for preparing a di-bromide of formula (ax) by brominating a corresponding compound of formula (ay):

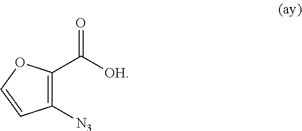

In one specific embodiment the invention provides a method for preparing a compound of formula (av):

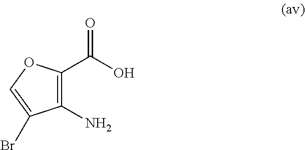

comprising reducing a corresponding nitro compound of formula (ba):

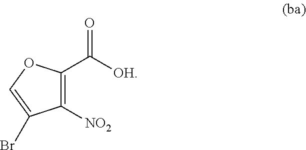

In one specific embodiment the invention provides a method for preparing a compound of formula (ba) by reducing a corresponding di-bromo compound of formula (bb):

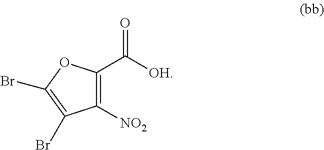

In one specific embodiment the invention provides a method for preparing a compound of formula (bb) by brominating a corresponding compound of formula (bc):

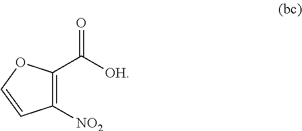

In one specific embodiment the invention provides a method for preparing a compound of formula (I):

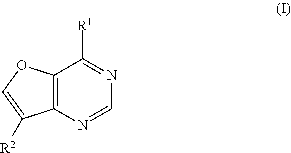

wherein:

R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;

n is 0-5;

R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; and

R² is a nucleoside sugar group;

comprising: converting a chloro compound of formula (bd):

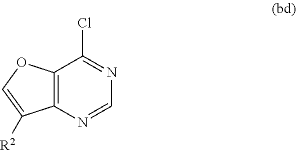

to the compound of formula (I).

In one specific embodiment the invention provides a method for preparing a compound of formula (bd) by cyclizing a corresponding compound of formula (be):

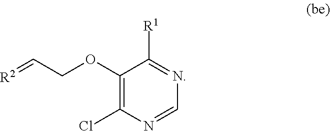

In one specific embodiment the invention provides a method for preparing a compound of formula (be) by reacting a compound of formula (bf) with a compound of formula (bg)

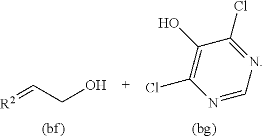

In one specific embodiment the invention provides a method for preparing a compound of formula (bg):

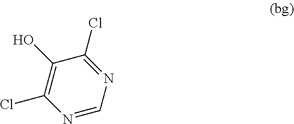

comprising removing a protecting group Rⱼ from a corresponding compound of formula (bh):

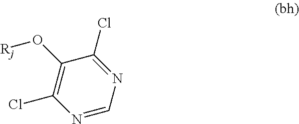

wherein Rⱼ is a hydroxy protecting group.

In one specific embodiment the invention provides a method for preparing a compound of formula (bh) by chlorinating a corresponding compound of formula (bi):

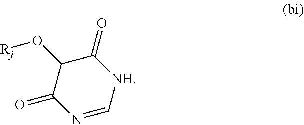

In one specific embodiment the invention provides a method for preparing a compound of formula (bi) by cyclizing a corresponding compound of formula (bj):

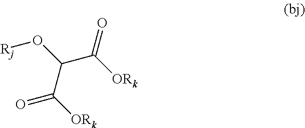

wherein each Rₖ is independently alkyl.

In one specific embodiment the invention provides a method for preparing a compound of formula (bj) by alkylating a corresponding compound of formula RⱼOH.

In one specific embodiment the invention provides a method for preparing a compound of formula (bf)

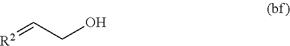

comprising reducing a corresponding compound of formula (bk):

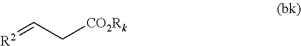

wherein Rₖ is alkyl.

In one specific embodiment the invention provides a method for preparing a compound of formula (bk) by reacting a compound of formula R²═O with a suitable double bond forming reagent.

In one specific embodiment the invention provides a method for preparing a compound of formula II:

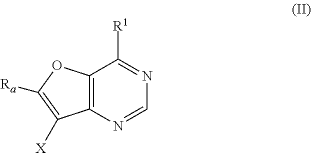

wherein:

R¹ is OR₃, SR₃, NR₃R₄, NR₃NR₄R₅, or aryl;

R₃, R₄, and R₅ are independently selected from the group consisting of H, alkyl and cycloalkyl;

X is H or Br; and

Rₐ is H or Si(CH₃)₃;

comprising: converting a corresponding chloro compound of formula (bm):

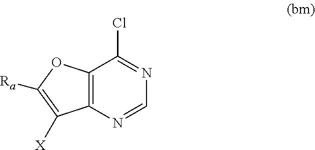

to the compound of formula (II).

In one specific embodiment the invention provides a method for preparing a compound of formula (bm) by chlorinating a corresponding compound of formula (bn):

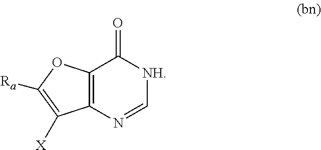

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula II: 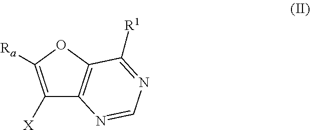
wherein:
R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;
n is 0-5;
R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;
Rₐ is H or Si(CH₃)₃; and
X is Br or I; or a protected analog thereof.

2. A compound of formula III: 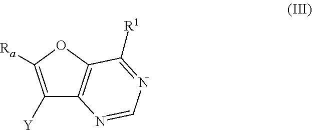
wherein:
R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;
n is 0-5;
R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;
Rₐ is H or Si(CH₃)₃; and
Y is a metallic group.

3. The compound of claim 2 wherein Y comprises Li, Cu, Zn, Cd, or Mg.

4. The compound of any one of claims 1-3 wherein R¹ is OR_{b} or NHR_{c}; wherein R_{b} is a hydroxy protecting group and R_{c} is an amino protecting group.

5. The compound of claim 4 wherein R_{b} is methoxymethyl, benzyloxymethyl, p-methoxybenzyloxymethyl, t-butoxymethyl, 2,2,2-trichloroethoxymethyl, tetrahydropyranyl, 1,4-dioxan-2-yl, tetrahydrofuranyl, 1-ethoxyethyl, 1-methyl-1-methoxymethyl, 2,2,2-trichloroethyl, t-butyl, allyl, p-methoxyphenyl, benzyl, p-methoxybenzyl, p-halobenzyl, 2,6-dichlorobenzyl, 2,4-dichlorobenzyl, triphenylmethyl, diphenylmethyl, 9-anthryl, trimethylsilyl, triethylsilyl, t-butyldimethylsilyl, t-butyldiphenylsilyl, diphenylmethylsilyl, formyl, acetyl, chloroacetyl, trichloroacetyl, trifluoroacetyl, pivaloyl, benzoyl, methoxycarbonyl, ethyloxycarbonyl, 2,2,2-trichloroethyloxycarbonyl, isobutoxycarbonyl, allyloxycarbonyl, vinyloxycarbonyl, or benzyloxycarbonyl.

6. The compound of claim 4 wherein is methoxycarbonyl, ethoxycarbonyl, 9-fluorenylmethoxycarbonyl, 2,2,2-trichloroethoxycarbonyl, t-butoxycarbonyl, vinyloxycarbonyl, allyloxycarbonyl, benzyloxycarbonyl, p-methoxybenzyloxycarbonyl, 2,4-dichlorobenzyloxycarbonyl, formyl, acetyl, piconyl, benzoyl, trichloroacetyl, or trifluoroacetyl.

7. The compound of any one of claims 1-6 wherein Rₐ is H.

8. A method for preparing a compound of formula IV: 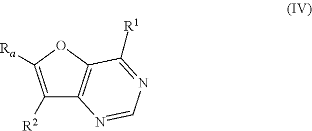
wherein:
R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;
R² is a nucleoside sugar group;
n is 0-5;
R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;
Rₐ is H; or a protected analog thereof;
comprising:
reacting a corresponding compound of formula III: 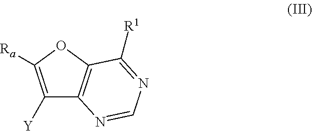
wherein Y is a metallic group, with an electrophilic nucleoside sugar group precursor; to provide the compound of formula (IV).

9. The method of claim 8 wherein the electrophilic nucleoside sugar group precursor is a compound of formula R²—Z; wherein R² is a nucleoside sugar group, and Z is a suitable leaving group.

10. The method of claim 8 wherein the electrophilic nucleoside sugar group precursor is a compound of formula R²(═O), wherein R² is a nucleoside sugar group.

11. A method for preparing a compound of formula IV: 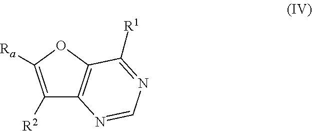
wherein:
R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;
R² is a nucleoside sugar group;
n is 0-5;
R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;
Rₐ is H; or a protected analog thereof;
comprising:
deoxygenating a corresponding compound of formula (V)

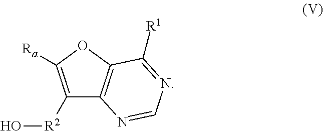

12. The method of claim 11 further comprising preparing the compound of formula (V) by reacting a corresponding compound of formula III: 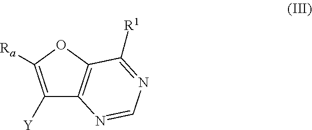
wherein: Y is a metallic group; or a protected analog thereof;
with an electrophilic nucleoside sugar group precurser of formula R²(═O),
wherein R² is a nucleoside sugar group.

13. The method of claim 11 further comprising preparing the compound of formula (V) by reacting a corresponding compound of formula III: 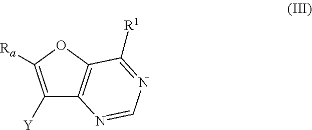
wherein: Y is a metallic group; or a protected analog thereof;
with an electrophilic nucleoside sugar group epoxide precurser of formula R²(>O), wherein R² is a nucleoside sugar group.

14. A compound of formula (V): 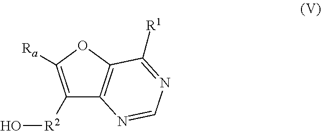
wherein:
R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;
n is 0-5;
R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic;
Rₐ is H; and
R² is a nucleoside sugar group.

15. A method for preparing a compound of formula IV: 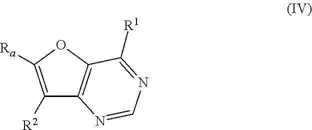
wherein:
R¹ is OR_{b} or NHR_{c};
R_{b} is a hydroxy protecting group;
R_{c} is an amino protecting group;
Rₐ is H; and
R² is a nucleoside sugar group;
comprising: reacting a corresponding compound of formula III: 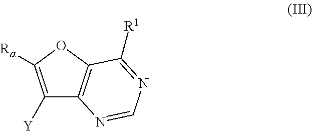
wherein Y is a metallic group, with an electrophilic nucleoside sugar group precurser; to provide the compound of formula (IV).

16. The method of claim 15 wherein the electrophilic nucleoside sugar group precursor is a compound of formula R²—Z; wherein R² is a nucleoside sugar group, and Z is a suitable leaving group.

17. The method of claim 15 wherein the electrophilic nucleoside sugar group precursor is a compound of formula R²(═O), wherein R² is a nucleoside sugar group.

18. A method for preparing a compound of formula IV: 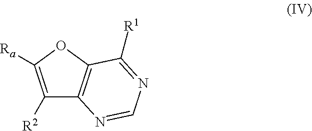
wherein:
R¹ is OR_{b} or NHR_{c};
R_{b} is a hydroxy protecting group;
R_{c} is an amino protecting group;
Rₐ is H; and
R² is a nucleoside sugar group;
comprising: deoxygenating a corresponding compound of formula (V)

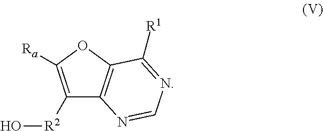

19. The method of claim 18 further comprising preparing the compound of formula (V) by reacting a corresponding compound of formula III: 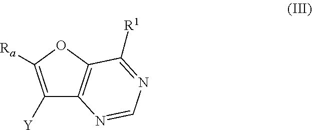
wherein:
Y is a metallic group;
with an electrophilic nucleoside sugar group precurser of formula R²(═O),
wherein R² is a nucleoside sugar group.

20. The method of claim 18 further comprising preparing the compound of formula (V) by reacting a corresponding compound of formula III: 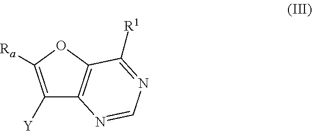
wherein:
Y is a metallic group;
with an electrophilic nucleoside sugar group epoxide precurser of formula R²(>O), wherein R² is a nucleoside sugar group.

21. A compound of formula (V): 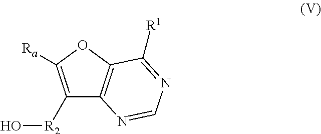
wherein:
R¹ is OR_{b} or NHR_{c};
R_{b} is a hydroxy protecting group;
R_{c} is an amino protecting group;
Rₐ is H; and
R² is a nucleoside sugar group.

22. A method for preparing a compound of the following formula (a): 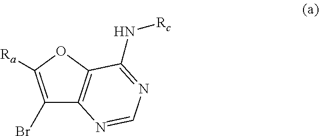
wherein Rₐ is H or Si(CH₃)₃, and R_{c} is an amino protecting group; comprising brominating a corresponding compound of formula (b):

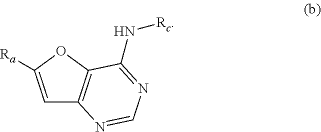

23. The method of claim 22 further comprising preparing the compound of formula (b) by protecting a corresponding amino compound of formula (c):

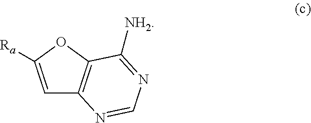

24. The method of claim 23 further comprising preparing the amino compound of formula (c) by converting a corresponding chloride of formula (d): 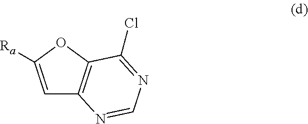
to the amino compound of formula (c).

25. The method of claim 24 further comprising preparing the chloro compound of formula (d) by chlorinating a corresponding compound of formula (e):

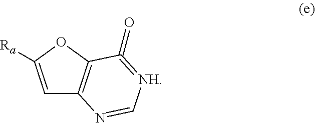

26. The method of claim 25 further comprising preparing the compound of formula (e) by cyclizing a corresponding compound of formula (f):

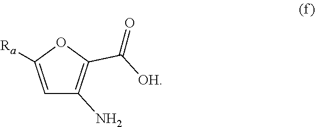

27. The method of claim 26 further comprising preparing the compound of formula (f) by reducing a corresponding azide of formula (g):

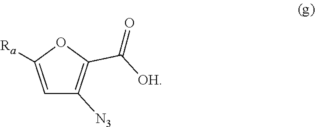

28. The method of claim 27 further comprising preparing the azide of formula (g) by converting a corresponding iodide of formula (h): 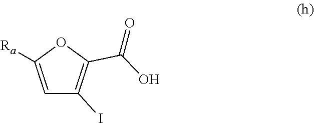
to the azide.

29. A method for preparing a compound of the following formula (i): 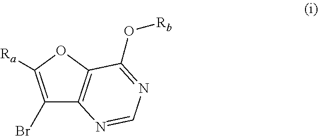
wherein Rₐ is H or Si(CH₃)₃, and R_{b} is a hydroxy protecting group; comprising brominating a corresponding compound of formula (j):

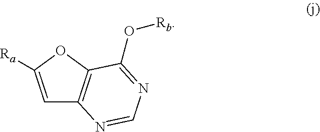

30. The method of claim 29 further comprising preparing the compound of formula (j) by protecting a corresponding compound of formula (e):

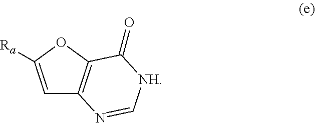

31. A method for preparing a compound of the following formula (k): 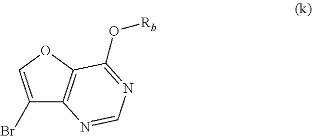
wherein R_{b} is a hydroxy protecting group comprising protecting a corresponding compound of formula (l):

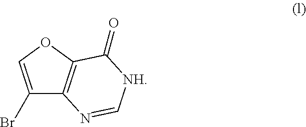

32. The method of claim 31 further comprising preparing compound of formula (l) by reducing a corresponding di-bromide of formula (m):

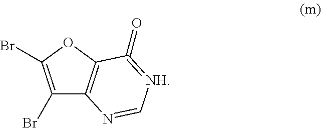

33. The method of claim 32 further comprising preparing compound of formula (m) by cyclizing a corresponding compound of formula (n):

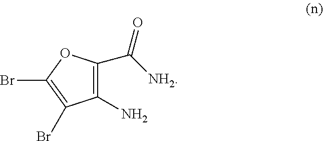

34. The method of claim 33 further comprising preparing compound of formula (n) by brominating a corresponding compound of formula (az):

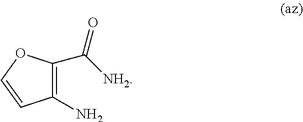

35. The method of claim 34 further comprising preparing compound of formula (az) by reducing a corresponding nitro compound of formula (o):

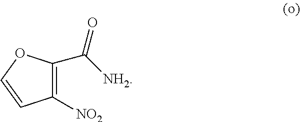

36. The method of claim 35 further comprising preparing compound of formula (o) by converting a corresponding ester of formula (p): 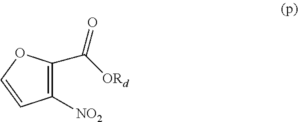
wherein R_{d} is alkyl to the compound of formula (o).

37. A method for preparing a compound of the following formula (k): 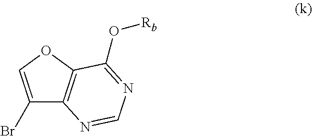
wherein R_{b} is a hydroxy protecting group comprising brominating a corresponding compound of formula (q):

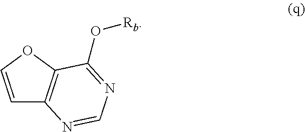

38. The method of claim 34 further comprising preparing the compound of formula (q) by protecting a corresponding compound of formula (r):

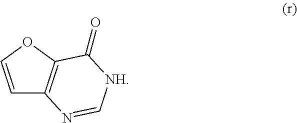

39. The method of claim 38 further comprising preparing the compound of formula (r) by cyclizing a corresponding compound of formula (s): 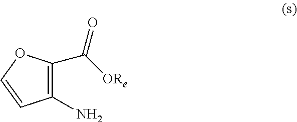
wherein Rₑ is alkyl.

40. A method for preparing a compound of the following formula (a): 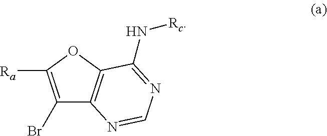
wherein Rₐ is H and R_{c} is an amino protecting group; comprising cyclizing a corresponding compound of formula (t):

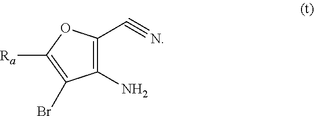

41. The method of claim 40 further comprising preparing the compound of formula (t) by reducing a corresponding azide of formula (u):

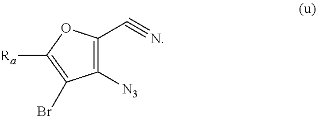

42. The method of claim 41 further comprising preparing the compound of formula (u) by reducing a corresponding dibromide of formula (v):

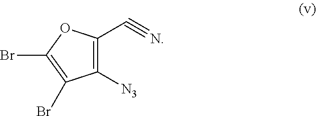

43. The method of claim 42 further comprising preparing the compound of formula (v) by brominating a corresponding compound of formula (w):

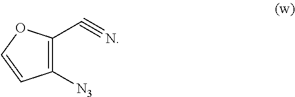

44. A method for preparing a compound of formula (s): 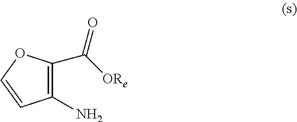
wherein Rₑ is alkyl comprising cyclizing a corresponding compound of formula (x):

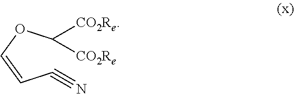

45. The method of claim 44 further comprising preparing the compound of formula (x) by alkylating a corresponding compound of formula (y):

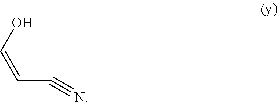

46. A method for preparing a compound of formula (t): 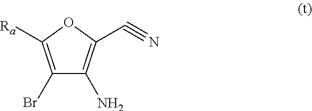
wherein R^{a} is hydrogen comprising reducing a corresponding dibromide of formula (z):

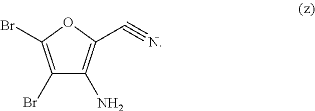

47. The method of claim 46 further comprising preparing the compound of formula (z) by brominating a corresponding compound of formula (aa):

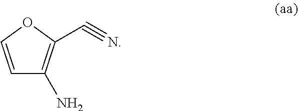

48. The method of claim 47 further comprising preparing the compound of formula (aa) by cyclizing a corresponding compound of formula (ab):

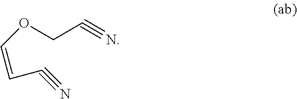

49. The method of claim 48 further comprising preparing the compound of formula (ab) by alkylating a corresponding compound of formula (y): 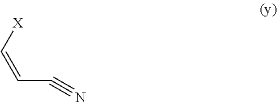
wherein X is Cl, Br, I, or OH.

50. A method for preparing a nitrile compound of formula (t): 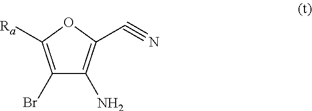
wherein Rₐ is hydrogen comprising converting a corresponding aldehyde of formula (ac): 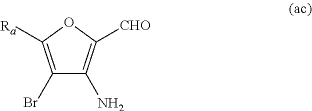
to the nitrile of formula (t).

51. The method of claim 50 further comprising preparing the aldehyde of formula (ac) by reducing a corresponding nitro compound of formula (ad):

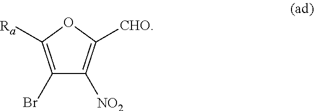

52. The method of claim 51 further comprising preparing the nitro compound of formula (ad) by reducing a corresponding dibromide of formula (ae):

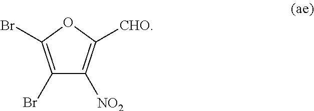

53. The method of claim 52 further comprising preparing the di-bromo compound of formula (ae) by nitrating a corresponding compound of formula (af):

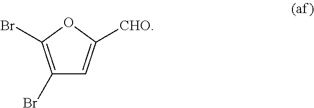

54. A method for preparing a compound of formula (l): 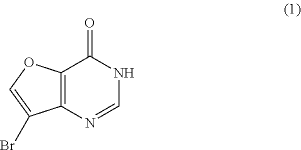
comprising cyclizing a corresponding compound of formula (ag): 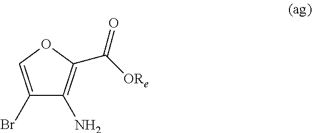
wherein Rₑ is alkyl.

55. The method of claim 54 further comprising preparing the compound of formula (ag) by converting an aldehyde of formula (ac): 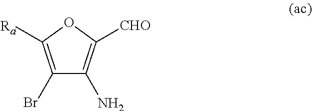
wherein Rₐ is hydrogen to the compound of formula (ag).

56. The method of claim 54 further comprising preparing the compound of formula (ag) by reducing a corresponding nitro compound of formula (ah):

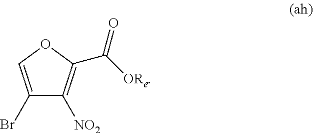

57. The method of claim 56 further comprising preparing the compound of formula (ah) by converting a corresponding aldehyde of formula (ad): 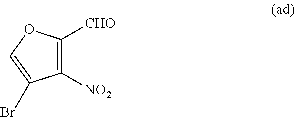
to the compound of formula (ah).

58. A method for preparing a compound of the following formula (a): 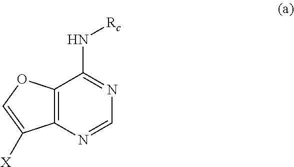
wherein R_{c} is an amino protecting group and X is Br or I; comprising protecting a corresponding compound of formula (ai):

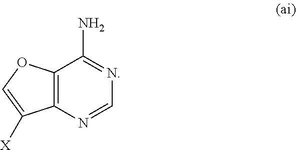

59. The method of claim 58 further comprising preparing the compound of formula (ai) by converting a corresponding acid of formula (aj): 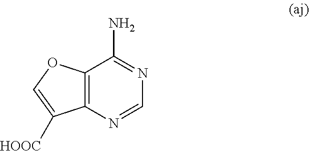
to the compound of formula (ai).

60. The method of claim 59 further comprising preparing the compound of formula (aj) by cyclizing a corresponding compound of formula (ak):

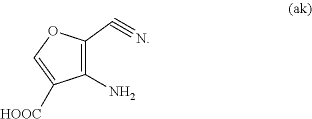

61. The method of claim 60 further comprising preparing the compound of formula (ak) by cyclizing a corresponding compound of formula (al):

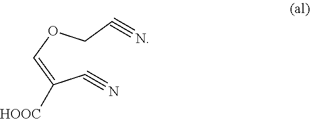

62. The method of claim 61 further comprising preparing the compound of formula (al) by converting a corresponding compound of formula (am): 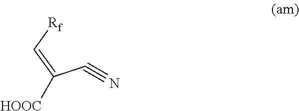
wherein R_{f} is Br, OH, or I to the compound of formula (am).

63. A method for preparing a compound of the following formula (i): 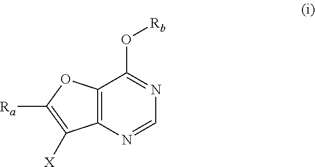
wherein Rₐ is H; X is Br or I; and R_{b} is a hydroxy protecting group; comprising converting a corresponding acid of formula (an) 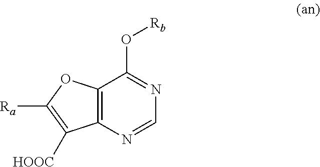
to the compound of formula (i).

64. The method of claim 63 further comprising preparing the acid of formula (an) by protecting a corresponding compound of formula (ao):

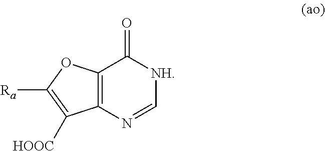

65. The method of claim 64 further comprising preparing the compound of formula (ao) by cyclizing a corresponding compound of formula (ap): 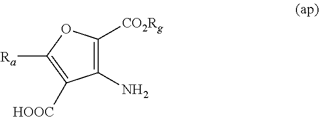
wherein R_{g} is alkyl.

66. The method of claim 65 further comprising preparing the compound of formula (ap) by cyclizing a corresponding compound of formula (aq):

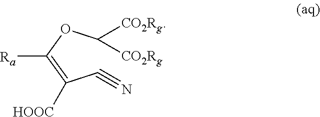

67. The method of claim 66 further comprising preparing the compound of formula (aq) by alkylating a corresponding compound of formula (am): 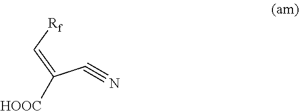
wherein R_{f} is OH.

68. A method for preparing a compound of formula (l): 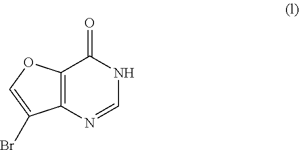
comprising reducing a corresponding di-bromide of formula (m):

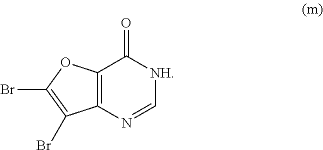

69. The method of claim 68 further comprising preparing the di-bromide of formula (m) by cyclizing a corresponding compound of formula (as): 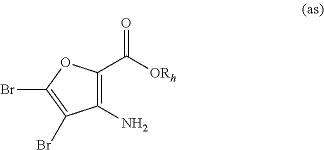
wherein Rₕ is alkyl.

70. The method of claim 69 further comprising preparing the compound of formula (as) by reducing a corresponding nitro compound of formula (at):

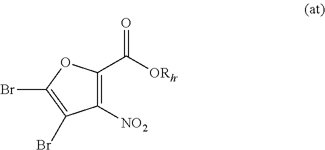

71. The method of claim 70 further comprising preparing the nitro compound of formula (at) by brominating a corresponding compound of formula (au):

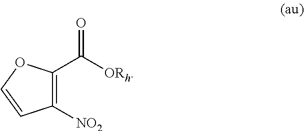

72. A method for preparing a compound of formula (l): 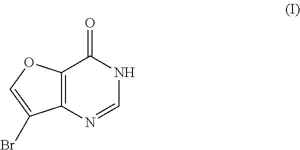
comprising cyclizing a corresponding compound of formula (av):

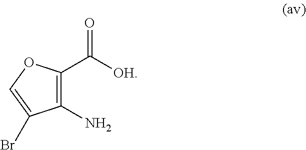

73. The method of claim 72 further comprising preparing the compound of formula (av) by reducing a corresponding azido compound of formula (aw):

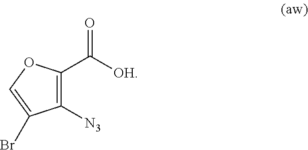

74. The method of claim 70 further comprising preparing the azido compound of formula (aw) by reducing a di-bromide of formula (ax):

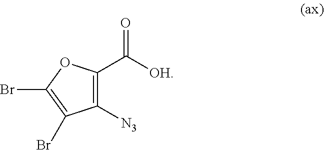

75. The method of claim 74 further comprising preparing the di-bromide of formula (ax) by brominating a corresponding compound of formula (ay):

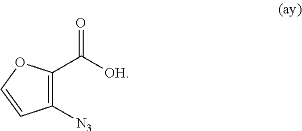

76. A method for preparing a compound of formula (av): 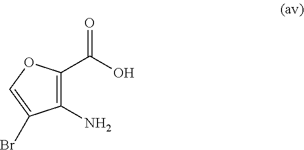
comprising reducing a corresponding nitro compound of formula (ba):

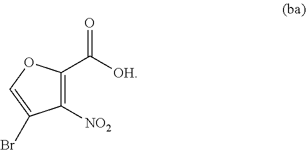

77. The method of claim 76 further comprising preparing the compound of formula (ba) by reducing a corresponding di-bromo compound of formula (bb):

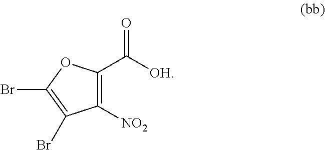

78. The method of claim 77 further comprising preparing the compound of formula (bb) by brominating a corresponding compound of formula (bc):

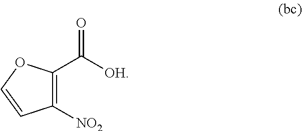

79. A method for preparing a compound of formula (I): 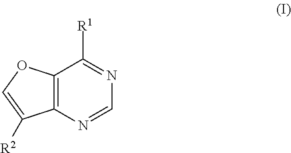
wherein:
R¹ is OR₃, SR₃, NR₃R₄, NR₃NR₄R₅, alkyl, substituted alkyl, alkenyl, substituted alkenyl, alkynyl, substituted alkynyl, aryl, substituted aryl, (CH₂)ₙ—CH(NHR₃)CO₂R₄, Cl, F, Br, I, CN, COOR₃, CONR₃R₄, NHC(═NR₃)NHR₄, NR₃OR₄, NR₃NO, NHCONHR₃, NR₃N═NR₄, NR₃N═CHR₄, NR₃C(O)NR₄R₅, NR₃C(S)NR₄R₅, NR₃C(O)OR₄, CH═N—OR₃, NR₃C(═NH)NR₄R₅, NR₃C(O)NR₄NR₅R₆, O—C(O)R₃, OC(O)—OR₃, ONH—C(O)O-alkyl, ONHC(O)O-aryl, ONR₃R₄, SNR₃R₄, S—ONR₃R₄, or SO₂NR₃R₄;
n is 0-5;
R₃, R₄, R₅, and R₆ are independently selected from the group consisting of H, alkyl, substituted alkyl, alkenyl, substituted alkenyl, alkynyl, substituted alkynyl, cycloalkyl, heterocyclic, aryl, substituted aryl, acyl, substituted acyl, SO₂-alkyl and NO; or R₃ and R₄ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; or R₄ and R₅ together with the nitrogen to which they are attached form a pyrrolidino, piperidino, piperazino, azetidino, morpholino, or thiomorpholino ring, which ring is optionally substituted with one or more substitutents independently selected from alkyl, substituted alkyl, alkoxy, substituted alkoxy, acyl, substituted acyl, acylamino, acyloxy, oxyacyl, amino, substituted amino, aminoacyl, aryl, substituted aryl, aryloxy, substituted aryloxy, cyano, halogen, hydroxyl, nitro, N₃, carboxyl, carboxyl esters, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, heteroaryl, substituted heteroaryl, heterocyclic, and substituted heterocyclic; and
R² is a nucleoside sugar group;
comprising:
converting a chloro compound of formula (bd): 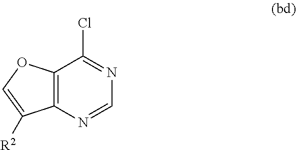
to the compound of formula (I).

80. The method of claim 79 further comprising preparing the compound of formula (bd) by cyclizing a corresponding compound of formula (be):

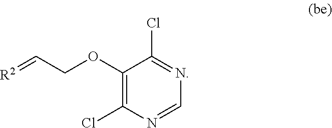

81. The method of claim 80 further comprising preparing the compound of formula (be) by reacting a compound of formula (bf) with a compound of formula (bg)

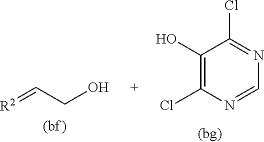

82. A method for preparing a compound of formula (bg): 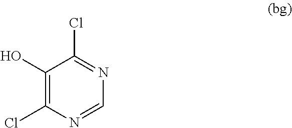
comprising removing a protecting group Rⱼ from a corresponding compound of formula (bh): 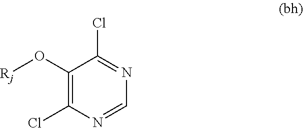
wherein Rⱼ is a hydroxy protecting group.

83. The method of claim 82 further comprising preparing the compound of formula (bh) by chlorinating a corresponding compound of formula (bi):

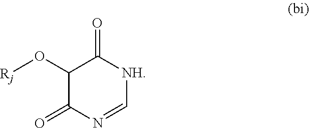

84. The method of claim 83 further comprising preparing the compound of formula (bi) by cyclizing a corresponding compound of formula (bj): 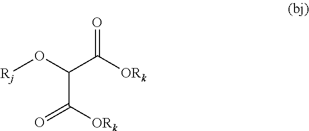
wherein each Rₖ is independently alkyl.

85. The method of claim 84 further comprising preparing the compound of formula (bj) by alkylating a corresponding compound of formula RⱼOH.

86. A method for preparing a compound of formula (bf) 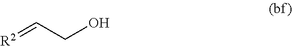
comprising reducing a corresponding compound of formula (bk): 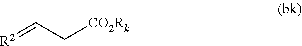
wherein Rₖ is alkyl.

87. The method of claim 86 further comprising preparing the compound of formula (bk) by reacting a compound of formula R²═O with a suitable double bond forming reagent.

88. A method for preparing a compound of formula II: 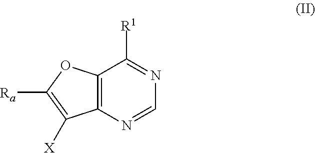
wherein:
R¹ is OR₃, SR₃, NR₃R₄, NR₃NR₄R₅, or aryl;
R₃, R₄, and R₅ are independently selected from the group consisting of H, alkyl and cycloalkyl;
X is H or Br; and
Rₐ is H or Si(CH₃)₃;
comprising:
converting a corresponding chloro compound of formula (bm): 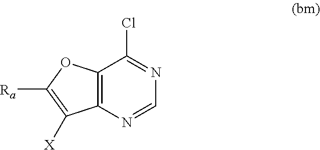
to the compound of formula (II).

89. The method of claim 88 further comprising preparing the compound of formula (bm) by chlorinating a corresponding compound of formula (bn):

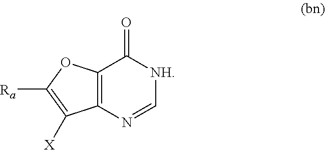

90. The method of any one of claims 8-11, 15-20, 79-81 and 86-87 or the compound of claim 14 or 21 wherein R² is a nucleoside sugar group as described in groups A-F hereinabove.

91. The method of any one of claims 8-11, 15-20, 79-81 and 86-87 or the compound of claim 14 or 21 wherein R² is a nucleoside sugar group of the formula:

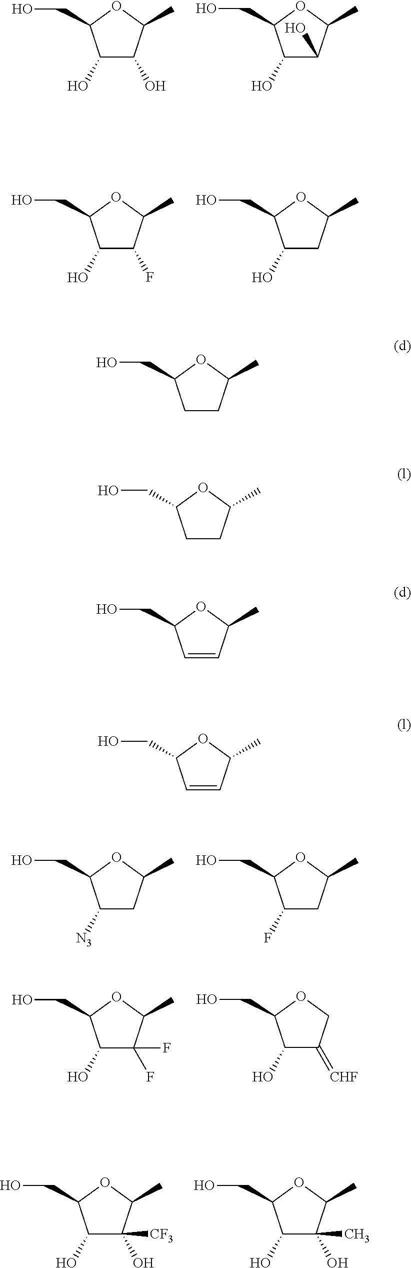

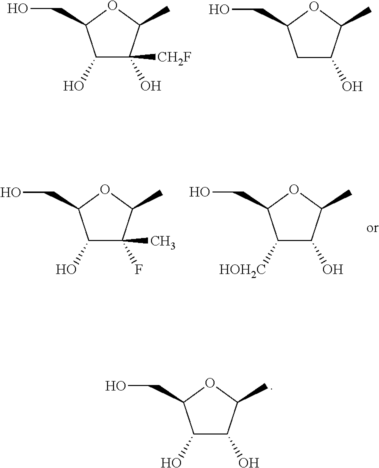

92. The method of any one of claims 8-11, 15-20, 79-81 and 86-87 or the compound of claim 14 or 21 wherein R² is a nucleoside sugar group of the formula

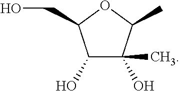

93. The method of claim 20 wherein the nucleoside sugar group epoxide precurser is a compound of formula: 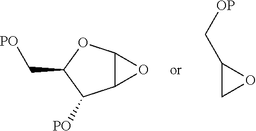
wherein each P is independently a protecting group.

94. The method of any one of claims 8-11, 15-20, 79-81 and 86-87 or the compound of claim 14 or 21 wherein R² is a nucleoside sugar group of the formula

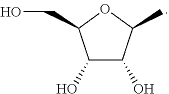

95. The method of claim 8, 12, 17, or 19 wherein the electrophilic nucleoside sugar group precurser is a compound of the following formula: 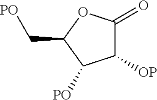
wherein each P independently a protecting group.

96. A novel compound as described herein.

97. A novel synthetic transformation described herein.
